(19) United States
(12) Patent Application Publication
(10) Pub. No.: US 2026/0106008 A1
(43) Pub. Date: Apr. 16, 2026

(54) Multimodal Recognition and Authentication of Pharmaceuticals

(71) Applicant: CVS Pharmacy, Inc., Woonsocket, RI (US)

(72) Inventors: Dwayne KURFIRST, Aurora, IL (US); Paul BARTRAM, Simpsonville, SC (US)

(21) Appl. No.: 18/913,448

(22) Filed: Oct. 11, 2024

(51) Int. Cl. G16H 20/10; G06N 20/00; G06V 10/764

(52) U.S. Cl. CPC G16H 20/10; G06N 20/00; G06V 10/764

(57) ABSTRACT

A method and system provide for automated counting of prescription product and enables virtual verification of the dispensed prescription product. The method and system include capturing by a camera at a first site an image of the prescription product to be dispensed according to a prescription to a patient, electronically displaying the image on a display at a second site remote from the first site, and electronically transmitting a verification from the second site to the first site in response to the image of the prescription product being determined at the second site to be consistent with the prescription.

### FIELD OF THE INVENTION

The present disclosure relates to the automated recognition and authentication of pharmaceuticals for managing supply chains, regulatory controls, prescriptions, and administration to patients. In particular, the present disclosure relates to devices and software to apply machine learning analytics to multimodal sensor data for recognizing and authenticating pharmaceuticals.

### BACKGROUND OF THE DISCLOSURE

Pharmaceutical supply chains and healthcare systems rely heavily on accurate identification and authentication of medications to ensure patient safety and regulatory compliance. Traditional methods of visual inspection and manual verification are prone to human error and can be time-consuming, especially when dealing with large volumes of pharmaceuticals. As the complexity and variety of medications continue to increase, there is a growing need for more sophisticated and reliable methods of pharmaceutical recognition and authentication.

Recent advancements in sensor technologies and machine learning algorithms have opened up new possibilities for automated pharmaceutical identification. However, many existing solutions still face challenges in accurately distinguishing between similar-looking medications or detecting counterfeit drugs. There is a need for more robust and comprehensive approaches that can leverage multiple types of sensory data to improve the accuracy and reliability of pharmaceutical recognition and authentication processes. New approaches may be particularly valuable for verification of pharmaceuticals from remote locations by enabling a computer system to make more precise verification decisions than would be practical for a pharmacist who does not have direct access to the dispensed medications.

Therefore, there still exists a need for a comprehensive multimodal system and method for accurately recognizing and authenticating pharmaceuticals using multiple types of sensory data and machine learning techniques.

### SUMMARY

Various aspects for multimodal recognition and authentication of pharmaceuticals are described. More particularly, systems and methods for capturing and analyzing multiple types of sensory data using machine learning techniques to accurately identify and verify pharmaceutical products are presented.

One general aspect includes a system that includes: a receptacle configured to receive a pharmaceutical product; a camera configured to capture image data for at least one image of the pharmaceutical product in the receptacle; at least one sensor configured to capture sensor data for at least one sensor signal based on the pharmaceutical product in the receptacle; at least one processor configured to: receive the image data and the sensor data; determine at least one visual feature from the image data; determine at least one sensor feature from the sensor data; apply at least one machine learning model to the at least one visual feature and the at least one sensor feature to determine a recognition confidence value for a pharmaceutical type; and return a recognition indicator based on the recognition confidence value for the pharmaceutical type.

Implementations may include one or more of the following features, or any combination thereof. The at least one visual feature may comprise a plurality of visual features selected from: a size feature; a color feature; a shape feature; and a marking feature; the at least one machine learning model may comprise a visual classifier model trained to recognize the plurality of visual features corresponding to the pharmaceutical type to generate a visual identifier confidence value; and the at least one processor may be further configured to use the visual identifier confidence value to determine the recognition confidence value. The at least one sensor may comprise a spectrometer configured to capture spectral data based on reflected light from a laser directed at the pharmaceutical product in the receptacle; the at least one sensor feature may comprise at least one spectral feature selected from: a spectrometer graph; and a signal-to-noise graph; the at least one machine learning model may comprise at least one classifier model selected from: a spectral classifier model trained to recognize the spectrometer graph corresponding to the pharmaceutical type to generate a spectral identifier confidence value; and a signal-to-noise classifier model trained to recognize the signal-to-noise graph corresponding to the pharmaceutical type to generate a signal-to-noise identifier confidence value; and the at least one processor may be further configured to use the spectral identifier confidence value or the signal-to-noise identifier confidence value to determine the recognition confidence value. The at least one sensor may comprise an olfactory sensor configured to capture olfactory data based on response to low-concentration chemicals suspended in air in the receptacle; the at least one sensor feature may comprise at least one olfactory feature corresponding to a presence of at least one volatile chemical present in the pharmaceutical type; the at least one machine learning model may comprise an olfactory classifier model trained to recognize the at least one olfactory feature corresponding to the pharmaceutical type to generate an olfactory identifier confidence value; and the at least one processor may be further configured to use the olfactory identifier confidence value to determine the recognition confidence value. The at least one sensor may comprise an audio sensor configured to capture audio data based on response to vibration of the pharmaceutical product in the receptacle; the at least one sensor feature may comprise at least one audio feature selected from: a vibration audio signal feature from controlled vibration of the pharmaceutical product; and a resonance frequency from a vibration frequency sweep of the pharmaceutical product; the at least one machine learning model may comprise an audio classifier model trained to recognize the at least one audio feature corresponding to the pharmaceutical type to generate an audio identifier confidence value; and the at least one processor may be further configured to use the audio identifier confidence value to determine the recognition confidence value. The system may further comprise: a vibration motor configured to controllably vibrate the receptacle using at least one selected frequency, wherein the at least one processor is further configured to selectively initiate the vibration motor to capture the audio data. The system may further comprise: a display in communication with the at least one processor and configured to display, responsive to the return of the recognition indicator by the at least one processor, a graphical user interface comprising the recognition indicator for the pharmaceutical product. The at least one sensor may comprise a plurality of sensors having different sensor types selected from: an image sensor; a spectrometer; an olfactory sensor; and an audio sensor; the at least one machine learning model may comprise a plurality of classifier models corresponding to the different sensor types; each classifier model of the plurality of classifier models: may correspond to a sensor type for that sensor of the plurality of sensors: and may be trained to return an identifier confidence value for the pharmaceutical type and that sensor type; and the at least one processor may be further configured to determine the recognition confidence value based on combining a plurality of identifier confidence values from the plurality of classifier models. The system may further comprise: a multimodal device comprising: the receptacle; the camera; the at least one sensor; and a control interface for a computing device; a first computing system at a first location and comprising: at least one processor configured to execute a pharmaceutical dispensing application for dispensing the pharmaceutical product based on a prescription; a peripheral interface configured for communication with the control interface of the multimodal device to control capture of the image data and the sensor data; and a first network interface configured for communication over a network; and a second computing system at a second location and comprising: at least one processor configured to execute a verification application for verifying dispensed pharmaceutical product against the prescription; a second network interface configured for communication over the network; and a display configured to display, responsive to the return of the recognition indicator by the at least one processor, a graphical user interface comprising the recognition indicator for the pharmaceutical product, wherein the verification application is configured to receive a verification input from a user in response to the display of the recognition indicator.

Another general aspect includes a method that includes: receiving, in a receptacle, a pharmaceutical product; capturing, using a camera, image data for at least one image of the pharmaceutical product in the receptacle; capturing, using at least one sensor, sensor data for at least one sensor signal based on the pharmaceutical product in the receptacle; determining, by at least one processor, at least one visual feature from the image data; determining, by the at least one processor, at least one sensor feature from the sensor data; determining, by the at least one processor applying at least one machine learning model to the at least one visual feature and the at least one sensor feature, a recognition confidence value for a pharmaceutical type; and returning, by the at least one processor, a recognition indicator based on the recognition confidence value for the pharmaceutical type.

Implementations may include one or more of the following features, or any combination thereof. The method may further comprise: determining, by the at least one processor, a visual identifier confidence value, wherein: the at least one visual feature comprise a plurality of visual features selected from: a size feature; a color feature; a shape feature; and a marking feature; the at least one machine learning model comprises a visual classifier model trained to recognize the plurality of visual features corresponding to the pharmaceutical type to generate the visual identifier confidence value; and determining the recognition confidence value is based on the visual identifier confidence value. The method may further comprise: determining, by the at least one processor, a spectral identifier confidence value or a signal-to-noise identifier confidence value, wherein: at least one sensor comprises a spectrometer configured to capture spectral data based on reflected light from a laser directed at the pharmaceutical product in the receptacle; the at least one sensor feature comprises at least one spectral feature selected from: a spectrometer graph; and a signal-to-noise graph; the at least one machine learning model comprises at least one classifier model selected from: a spectral classifier model trained to recognize the spectrometer graph corresponding to the pharmaceutical type to generate the spectral identifier confidence value; and a signal-to-noise classifier model trained to recognize the signal-to-noise graph corresponding to the pharmaceutical type to generate the signal-to-noise identifier confidence value; and determining the recognition confidence value is based on the spectral identifier confidence value or the signal-to-noise identifier confidence value. The method may further comprise: determining, by the at least one processor, an olfactory identifier confidence value, wherein: the at least one sensor comprises an olfactory sensor configured to capture olfactory data based on response to low-concentration chemicals suspended in air in the receptacle; the at least one sensor feature comprises at least one olfactory feature corresponding to a presence of at least one volatile chemical present in the pharmaceutical type; the at least one machine learning model comprises an olfactory classifier model trained to recognize the at least one olfactory feature corresponding to the pharmaceutical type to generate the olfactory identifier confidence value; and determining the recognition confidence value is based on the olfactory identifier confidence value. The method may further comprise: determining, by the at least one processor, an audio identifier confidence value, wherein: the at least one sensor comprises an audio sensor configured to capture audio data based on response to vibration of the pharmaceutical product in the receptacle; the at least one sensor feature comprises at least one audio feature selected from: a vibration audio signal feature from controlled vibration of the pharmaceutical product; and a resonance frequency from a vibration frequency sweep of the pharmaceutical product; the at least one machine learning model comprises an audio classifier model trained to recognize the at least one audio feature corresponding to the pharmaceutical type to generate the audio identifier confidence value; and determining the recognition confidence value is based on the audio identifier confidence value. The method may further comprise: controllably vibrating, using a vibration motor, the receptacle using at least one selected frequency to initiate capture of the audio data. The method may further comprise: displaying, on a display in communication with the at least on processor and responsive to the return of the recognition indicator by the at least one processor, a graphical user interface comprising the recognition indicator for the pharmaceutical product. The method may further comprise: determining a plurality of identifier confidence values using a plurality of classifier models, wherein: the at least one sensor comprises a plurality of sensors having different sensor types selected from: an image sensor; a spectrometer; an olfactory sensor; and an audio sensor; the at least one machine learning model comprises a plurality of classifier models corresponding to the different sensor types; each classifier model of the plurality of classifier models: corresponds to a sensor type for that sensor of the plurality of sensors: and is trained to return an identifier confidence value for the pharmaceutical type and that sensor type; and combining the plurality of identifier confidence values from the plurality of classifier models to determine the recognition confidence value. The method may further comprise: receiving, by a first computing system at a first location, a prescription; dispensing, prior to receiving the pharmaceutical product in the receptacle, the pharmaceutical product based on the prescription; receiving, by the first computer system and from a multimodal device, the image data and the sensor data, wherein the multimodal device comprises: the receptacle; the camera; the at least one sensor; receiving, over a network and by a second computing system at a second location, the recognition indicator; displaying, on a display of the second computing system, a graphical user interface comprising the recognition indicator for the pharmaceutical product; and receiving, by the second computing system and from a user, a verification input for the pharmaceutical product corresponding to the prescription in response to the display of the recognition indicator.

Still another general aspect includes a device that includes: a receptacle configured to receive a pharmaceutical product; a camera configured to capture image data for at least one image of the pharmaceutical product in the receptacle; at least one sensor configured to capture sensor data for at least one sensor signal based on the pharmaceutical product in the receptacle; and a control interface to at least one processor configured to: receive the image data and the sensor data; determine at least one visual feature from the image data; determine at least one sensor feature from the sensor data; apply at least one machine learning model to the at least one visual feature and the at least one sensor feature to determine a recognition confidence value for a pharmaceutical type; and return a recognition indicator based on the recognition confidence value for the pharmaceutical type.

Implementations may include one or more of the following features, or any combination thereof. The at least one sensor may comprise a plurality of sensors having different sensor types selected from: an image sensor; a spectrometer; an olfactory sensor; and an audio sensor; the at least one machine learning model may comprise a plurality of classifier models corresponding to the different sensor types; each classifier model of the plurality of classifier models: may correspond to a sensor type for that sensor of the plurality of sensors: and may be trained to return an identifier confidence value for the pharmaceutical type and that sensor type; and the at least one processor may be further configured to determine the recognition confidence value based on combining a plurality of identifier confidence values from the plurality of classifier models.

The various examples advantageously apply the teachings of automated systems for recognizing and authenticating pharmaceuticals to improve the functionality of such computer systems. The various embodiments include operations to overcome or at least reduce the issues previously encountered in automated recognition and authentication of pharmaceuticals and, accordingly, are more reliable and/or efficient than other computing systems configured for similar purposes and/or integrated into particular machines configured for this purpose. That is, the various embodiments disclosed herein include hardware and/or software with functionality to improve automated pharmaceutical recognition and authentication, such as by using multimodal configurations of sensors and a corresponding network of machine learning models to more accurately identify target pharmaceuticals in a single operation. Accordingly, the embodiments disclosed herein provide various improvements to automated systems for pharmaceutical recognition and authentication.

It should be understood that language used in the present disclosure has been principally selected for readability and instructional purposes, and not to limit the scope of the subject matter disclosed herein. All examples and features mentioned above can be combined in any technically possible way.

### BRIEF DESCRIPTION OF THE DRAWINGS

The present disclosure is illustrated by way of example, and not by way of limitation in the figures of the accompanying drawings in which like reference numerals are used to refer to similar elements.

FIG. 1 schematically illustrates a pharmaceutical analysis system, according to aspects of the present disclosure.

FIG. 2A is a flowchart of an example method for multi-modal analysis of a target object.

FIG. 2B schematically illustrates a system for multimodal identification and confidence calculation supporting the example method of FIG. 2A.

FIG. 3 is a flowchart of an example method for training multiple classifier models for drug recognition.

FIG. 4 schematically illustrates a pharmaceutical verification and dispensing system.

FIGS. 5A, 5B, and 5C illustrate different views of an example multimodal device.

FIG. 6 schematically illustrates a multimodal analysis system and associated method configured for a wireless multimodal device.

FIG. 7 schematically illustrated a multimodal analysis system and associated method configured for volume automated processing.

FIG. 8 schematically illustrates an example computing system.

### DETAILED DESCRIPTION

The present disclosure relates to a system and method for multimodal recognition and authentication of pharmaceuticals. In some examples, the system may include a receptacle designed to receive a pharmaceutical product. A camera may be configured to capture image data of the pharmaceutical product within the receptacle. The system may also include one or more sensors designed to capture sensor data based on the pharmaceutical product in the receptacle. These sensors may include, for example, a spectrometer, an olfactory sensor, an audio sensor, or any combination thereof.

In some examples, the system may include a processor configured to receive the image data and the sensor data. The processor may be further configured to determine visual features from the image data and sensor features from the sensor data. For example, the visual features may include size, color, and shape of the pharmaceutical product, while the sensor features may include spectral data, olfactory data, and audio data corresponding to the pharmaceutical product.

In some examples, the system may apply machine learning models to the visual and sensor features to determine a recognition confidence value for a pharmaceutical type. The machine learning models may include, for example, a visual classifier model, a spectral classifier model, an olfactory classifier model, an audio classifier model, or any combination thereof. Each of these classifier models may be trained to recognize features corresponding to a specific pharmaceutical type and generate an identifier confidence value.

In some examples, the system may return a recognition indicator based on the recognition confidence value for the pharmaceutical type. This recognition indicator may provide a measure of confidence that the pharmaceutical product is of a certain type, which may be useful in various applications such as managing supply chains, regulatory controls, prescriptions, and administration to patients.

In some examples, the multimodal device may be housed in a single container that might be worn around the wrist and then send feedback to a device such as a tablet, mobile phone, or laptop, through Bluetooth. The system may automatically analyze individual pills from a batch. In some configurations, the system may require specific conditions for accurate analysis, such as correct color hue lighting conditions for the camera, close proximity of the sensors to the pill, or specific preparation of the pill for certain sensors, such as pulverizing and/or dissolving the pill in a base liquid for some infrared (IR) spectrum or olfactory sensors.

In some examples, the system may use a database of pill pictures, IR spectrum data, sound data, and olfactory data for baseline models of pharmaceutical targets to be identified. These baseline models may be used for preliminary testing and then the system can test against these baseline models for accurate recognition and authentication of pharmaceuticals.

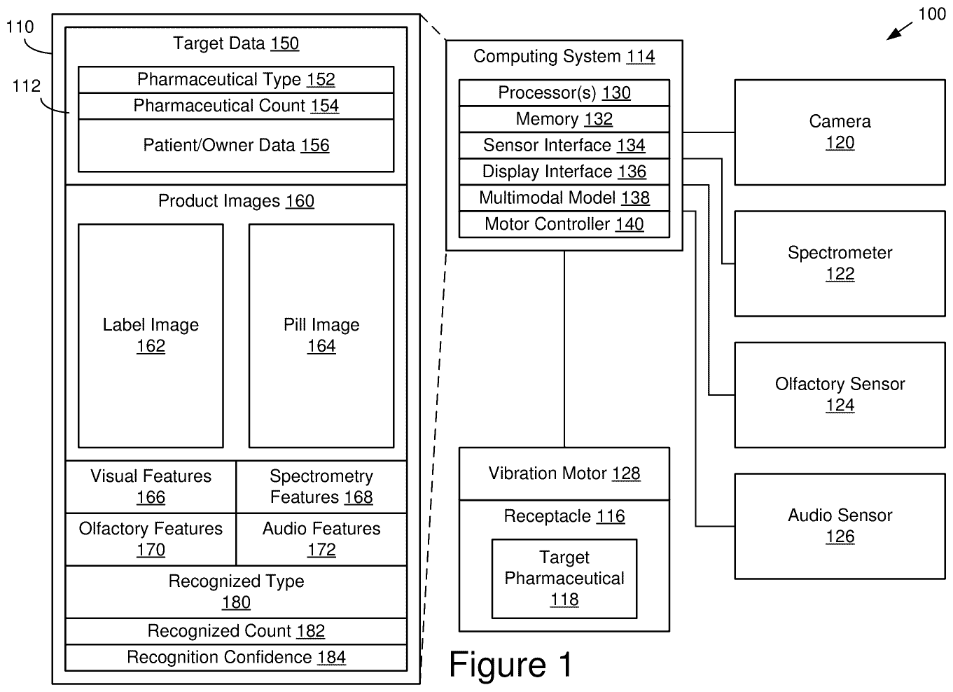

Referring to FIG. 1, a pharmaceutical analysis system 100 may include a display device 110 configured to provide a graphical user interface (GUI) 112 for a comparing system 114 configured to analyze a pharmaceutical product, such as target pharmaceutical 118, in a receptacle 116. Receptacle 116 may be designed to hold the pharmaceutical product during the analysis process. In some cases, the receptacle 116 may be adjustable to accommodate pharmaceutical products of various sizes and shapes.

System 100 may also include a camera 120 configured to capture image data for at least one image of the pharmaceutical product in receptacle 116. Camera 120 may be positioned to capture images from a fixed position to provide a single view and corresponding image and/or adjustable to various angles, providing a comprehensive view of the pharmaceutical product for multiple images. In some cases, camera 120 may be equipped with specialized lenses or filters to enhance the image quality or to capture specific visual features of the pharmaceutical product. Camera 120 may be a digital camera comprised of at least one image sensor and a corresponding digital processor and encoding scheme for capturing and encoding a digital image of the pharmaceutical product in receptacle 116.

System 100 may include at least one sensor configured to capture sensor data for at least one sensor signal based on the pharmaceutical product in receptacle 116. The sensor may include a spectrometer 122, an olfactory sensor 124, an audio sensor 126, or any combination thereof. Spectrometer 122 may be configured to capture spectral data based on reflected light from the pharmaceutical product. Olfactory sensor 124 may be designed to capture olfactory data based on the response to low-concentration chemicals suspended in the air in receptacle 116. Audio sensor 126 may be configured to capture audio data based on the response to vibration of the pharmaceutical product in the receptacle 116, such as vibration induced by vibration motor 128. In some configurations, system 100 may include at least one of each sensor type to provide multimodal analysis of image data, spectral data, olfactory data, and audio data in parallel for target pharmaceutical 118.

In some cases, system 100 may include a processor 130 configured to receive the image data and the sensor data. Processor 130 may be a high-speed processing unit capable of handling large volumes of data and performing complex calculations. Processor 130 may be configured to determine visual features from the image data and sensor features from the sensor data. For example, the visual features may include size, color, and shape of the pharmaceutical product, while the sensor features may include spectral data, olfactory data, and audio data corresponding to the pharmaceutical product. In some configurations, processor 130 may be configured to execute a multimodal analysis engine based on multimodal model 138 and the collected visual and other sensor features from the corresponding sensor data.

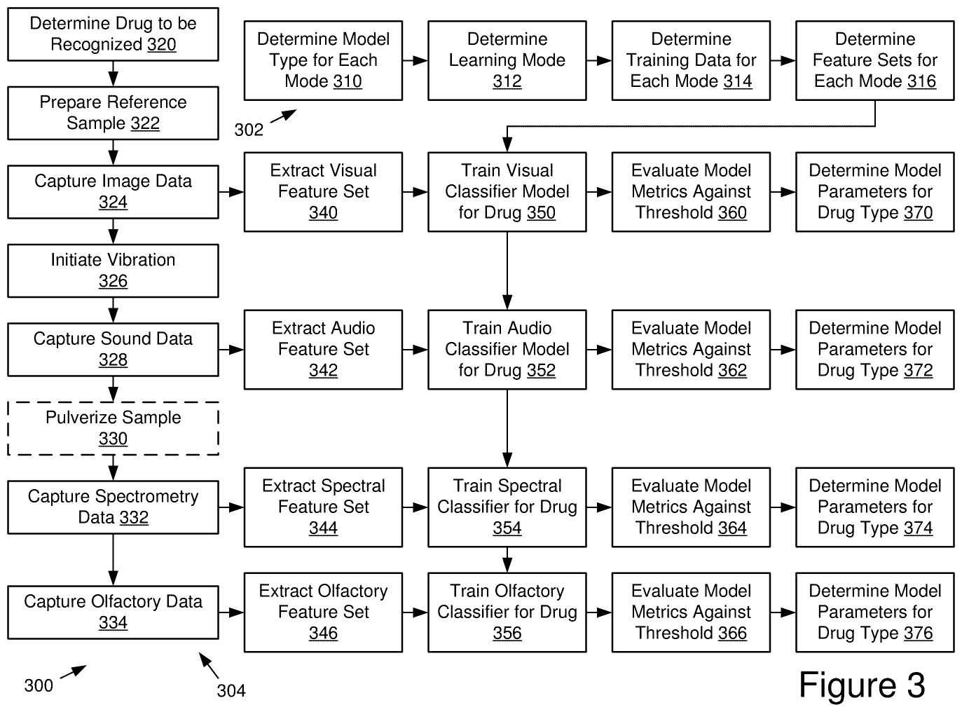

System 100 may apply machine learning models to the visual and sensor features to determine a recognition confidence value for a pharmaceutical type. The machine learning models may include a visual classifier model, a spectral classifier model, an olfactory classifier model, an audio classifier model, or any combination thereof. Each of these classifier models may be trained to recognize features corresponding to a specific pharmaceutical type and generate an identifier confidence value. In some configurations, these different models may be integrated into multimodal model 138 that may be executed by computing system 114 to generate the recognition confidence value for display as recognition confidence indicator 184. An example multimodal analysis engine 410, such as may be implemented by computing system 114, is further described below with regard to FIG. 4. System 100 may return a recognition indicator, such as recognized type indicator 180, based on the recognition confidence value for the pharmaceutical type. This recognition indicator may provide a measure of confidence that the pharmaceutical product is of a certain type, which may be useful in various applications such as managing supply chains, regulatory controls, prescriptions, and administration to patients.

In some examples, spectrometer 122 may be configured to capture spectral data based on reflected light from a laser directed at target pharmaceutical 118 in receptacle 116. Spectrometer 122 may be designed to analyze the infrared spectrum of the pharmaceutical product, providing a unique spectral signature that can be used for identification and authentication. An infrared sensor positioned to receive the reflected laser from the pharmaceutical product may generate a set of spectral data as an electrical signal to the computer system. The spectral data may include information about the chemical composition of the pharmaceutical product, which can be used to verify its authenticity and identify any potential contaminants or adulterants.

In some examples, system 100 may also include an olfactory sensor 124. Olfactory sensor 124 may be designed to capture olfactory data based on the response to low-concentration chemicals suspended in the air in receptacle 116. Olfactory sensor 124 may function similarly to a human nose, detecting volatile organic compounds (VOCs) that are released by the pharmaceutical product. These VOCs may provide a unique olfactory signature for the pharmaceutical product, which can be used for identification and authentication. An artificial olfactory sensor may use bioelectric, chemical, and/or electrochemical reactions to specific VOCs in the receptacle. For example, an array of electrochemically responsive sensor components for specific VOCs that are common among pharmaceutical products may be arranged in a circuit that generates an electrical signature based on the reactivity of the various VOCs. Sensor subcomponents may include an array of metal oxide based electrochemical sensors, conductive polymer sensors, biomimetic biosensors, and organic dye-based colorimetric sensors. This electrical signature may provide the olfactory data used by computer system 114.

In some examples, system 100 may further include an audio sensor 126. Audio sensor 126 may be configured to capture audio data based on the response to vibration of target pharmaceutical 118 in receptacle 116. Different pharmaceutical products may respond differently to vibration and return different acoustic signals based on their physical properties, such as its size, shape, density, and material composition. In some configurations, the frequency used to stimulate the target pharmaceutical may be varied for different sample frequencies and corresponding acoustic responses to construct the audio data set for analysis. A frequency sweep across a range of frequencies using a known step size may generate an audio spectrum from which a resonance frequency may be determined. The resonance frequencies of the pharmaceutical products can provide a physical characteristic that is uncommon among different pharmaceuticals and may be used for identification and authentication. In some cases, system 100 may include a vibration motor 128. Vibration motor 128 may be configured to controllably vibrate receptacle 116 using at least one selected frequency. Vibration motor 128 may be used to induce vibrations in the target pharmaceutical 118 at known frequencies, which can then be detected by the audio sensor 126. Vibration motor 128 may be controlled by processor 130, which may selectively initiate the vibration motor 128 to capture the audio data.

Processor 130 may be configured to receive the image data and the sensor data, determine visual features from the image data and sensor features from the sensor data, apply machine learning models to the visual and sensor features to determine a recognition confidence value for a pharmaceutical type, and return a recognition indicator based on the recognition confidence value for the pharmaceutical type. In order to collect this data from a variety of sensor types, such as camera 120, spectrometer 122, olfactory sensor 124, and audio sensor 126, computing system 113 may include a plurality of physical interfaces and corresponding signal protocols for receiving data as electrical signals from the various sensors. For example, computing system 114 may include a sensor interface 134 configured to connect processor 130 and memory 132 to the various sensors for receiving corresponding sensor data. Sensor interface 134 may include a peripheral computing interface, such as universal serial bus (USB), peripheral component interface express (PCIe), or a similar interface for receiving binary data from the various sensors using established computing interface standards. In other configurations, computing system 114 may include a bus, such as a system management bus, comprised of a set of conductors for transmitting electrical signals among various components of the computing system and one or more of the sensors may be directly integrated for communication via the bus. For example, one or more sensors may be embodied in a microchip or integrated circuit package configured for communication via the bus. Sensor interface 134 may include pathways and protocols for control signals to the various sensors to initiate data collection, in addition to receiving resulting data signals. Some sensor components may rely on or integrate with a separate component for initiating data collection. For example, audio sensor 126 may be dependent on the actuation of vibration motor 128 to receive acoustic signals from target pharmaceutical 118. Computing system 114 may include one or more controllers, such as motor controller 140, for controlling these other components, such as vibration motor 128. Motor controller 140 may use similar interface methods, such as peripheral or bus communication, to control the corresponding devices. In another example, spectrometer 122 may include or respond to a laser that is separately controlled by computing system 114.

Processor 130 may include one or more processors, such as microprocessors, central processing units (CPUs), or specialized processor circuits, configured to operate alone or in combination to execute the functions of computing system 114. For example, processor 130 may include the CPU of a general computing device, such as a mobile phone, tablet, laptop, personal computer, or similar device, and may include one or more processing cores configured to execute instructions stored in memory 132. Memory 132 may include one or more of volatile memory and non-volatile memory (e.g., random access memory (RAM), read-only memory (ROM), flash memory, hard disk, optical disk, etc.). It should be understood that memory 132 may be a single device or may include multiple types of devices and configurations. Processor 130 may be configured to receive image data and sensor data from camera 120, spectrometer 122, olfactory sensor 124, and audio sensor 126 for processing through multimodal model 138. Image data, spectral data, olfactory data, and audio data may be stored in short-term or long-term memory locations in memory 132 for processing by processor 130.

Processor 130 may apply machine learning models to the visual and sensor features in the received camera and sensor data to determine a recognition confidence value for a pharmaceutical type. For example, computing system 114 may include multimodal model 138 to use these disparate data types and sources to make an overall determination of the pharmaceutical type with a stated level of confidence. The machine learning models may include, for example, a visual classifier model, a spectral classifier model, an olfactory classifier model, an audio classifier model, or any combination thereof. Each of these classifier models may be trained to recognize features corresponding to a specific pharmaceutical type and generate a confidence value for that pharmaceutical type. Each sensor may generate a different pattern of high-dimensional pattern data that may be processed through a combination of statistical pattern analysis and machine learning model analysis, and may also reflect a set of logical rules (such as using Boolean logic) to determine among various processing paths for combining the results of discrete sensor data. In some configurations, statistical pattern analysis methods may include linear analysis, principle component analysis (PCA), linear discriminant analysis, and support vector machine. For example, one or more statistical models may be applied to raw sensor signal data to provide one or more data features for input to corresponding machine learning models.

Machine learning models may include artificial neural networks (ANN), multilayer perceptron (MLP), and k-nearest networks (KNN). In some configurations, each classifier model may be based on an artificial neural network having a topology based on the feature set for the particular sensor data and a set of node weights trained using corresponding reference data for each pharmaceutical target. For verification, a single classifier of each type may be trained for each pharmaceutical target and grouped into sets of classifiers for each sensor type corresponding the verification target. For broader recognition applications, multi-classifiers may be trained on a set of similar pharmaceutical targets and corresponding pharmaceutical types or single target classifiers may be processed in parallel or sequence. The set of classifiers may each return a pharmaceutical type (class) and corresponding confidence value. Each classifier may use a corresponding set of features extracted from the corresponding sensor data. For example, a set of visual features may be extracted from the image data, a set of spectrometry or spectral features may be extracted from the spectral data, a set of olfactory features may be extracted from the olfactory data, and a set of audio features may be extracted from the audio data. For more complex analysis, multiple feature sets and classifier models may sequentially refine features for the classification decision. For example, the visual classifier model may include a combination of shape, size, and color classifiers operating on different and/or overlapping features extracted from the same image data.

Multimodal model 138 may be an aggregate model that receives features and/or classification types and confidence values from the different sensor-specific classifier models to determine an aggregate classification type and corresponding recognition confidence. Multimodal model 138 may itself be a neural network model trained based on a training set of classifier data for each target pharmaceutical in the set of targets for which system 100 is configured. For example, the pharmaceutical type and confidence value for each sensor type, may be the features input to multimodal model 138 to determine an aggregate recognized pharmaceutical type and corresponding confidence value. In some configurations, multimodal model 138 may be based on statistical functions, such as voting functions or weighted average calculations. Additional layers of logical rules may also be used for eliminating edge cases and/or determining errors in the classification process indicating inconclusive or unreliable results.

Computing system 114 may return a recognition indicator based on the recognition confidence value for the pharmaceutical type. This recognition indicator may be a composite recognized type and recognition confidence based on the combination of the sensor classifiers through multimodal model 138. This recognition indicator may provide a measure of confidence that the pharmaceutical product is of a certain type, which may be useful in various applications such as managing supply chains, regulatory controls, prescriptions, and administration to patients. Computing system 114 may output the recognition indicator and related information to a user through graphical user interface 112 on display device 110.

System 100 may further include display device 110 in communication with processor 130. Display device 110 may include a monitor, screen, or display unit integrated with or connected to computing system 114. For example, computing system 114 may be embodied in a mobile phone or tablet computer and display device 110 may be the light emitting diode (LED) screen of that device. In some configurations, display device 110 may be a separate but communicatively coupled device and computing system 114 may include a display interface 136 for controlling what is displayed on the display device. For example, computing system 114 may be a specialized multimodal device and communicate with a mobile phone or tablet computer as the display device using a wired (e.g., USB) or wireless (e.g., Bluetooth or WiFi) connection through display interface 136. Display device 110 may be configured to display a graphical user interface 112 to automatically output recognition or authentication data to a user. Graphical user interface 112 may display various types of information, including the recognition indicator for the pharmaceutical product. This may allow a user to easily understand the results of the analysis performed by the system 100.

Graphical user interface 112 may provide a visual interface for users to interact with and monitor pharmaceutical analysis system 100. It may display several key components including target data 150, product images 160, feature indicators for one or more features extracted from each sensor data source, such as visual features 166, spectrometry features 168, olfactory features 170, and audio features 172, as well as recognition indicators for recognized type 180, recognized count 182, and recognition confidence 184. Users may interact with the graphical user interface 112 to select or input target data 150 for authentication, view intermediate data related to the analysis, such as product images and feature indicators, view recognition analysis results, and make decisions based on the displayed information. For example, a caregiver may determine whether the medication in receptacle 116 is safe to give a patient, a pharmacist may determine whether the medication in receptacle 116 matches a prescription being filled and/or indicated on a product label, or an inspector may determine whether one or more specimens from a batch of pharmaceuticals matches manufacturing specifications, a shipping manifest, border control documentation, or other authentication. Similar technology may be applied to automated pill identification/sorting/counting applications for medication returns or other organization.

Target data 150 may display overview information about the pharmaceutical product being analyzed. For example, for many applications, system 100 may be employed to see whether a target pill matches the pharmaceutical it is supposed to be, such as for verification before administering to a patient or dispensing a prescription. Target data 150 may include a pharmaceutical type 152, which may specify the expected drug or medication, such as the drug's name (proprietary and/or generic) and dose. Pharmaceutical count 154 may indicate the number of units expected based on the dosing or prescription fill. Patient or owner data 156 may contain relevant information about the individual associated with the prescription or pharmaceutical product (for drug administration or prescription fill) or an individual or organization that owns or possesses the target pharmaceuticals (for substance control applications). Target data 150 may be populated based on one or more data sources related to the application, such as patient or prescription records from a medical record management system or pharmacy computing system. In some configurations, computing system 114 may include one or more target records selectable by the user for populating target data 150 for each authentication operation.

Product images 160 may offer visual representations of the analyzed pharmaceutical. Label image 162 may display the packaging or labeling of the product, while the pill image 164 may show a close-up view of the actual pharmaceutical unit. In some configurations, product images 160 may be reference images that support target data 150 and user verification that the labels and pills being authenticated generally match what is in the product images. For example, pharmaceutical product records indexed by pharmaceutical type 152 may include such reference images and/or multimodal model 138 may use reference images in the classification and recognition of visual features from the image data. In some configurations, product images 160 may be dynamically generated images of target pharmaceutical 118 at the time of the authentication. Product images 160 may be displayed to the user for their reference and/or for checking positioning of the target and quality of the image captured. In some configurations, computing system 114 may include processing of image data to generate error messages for images with poorly positioned pills in receptacle 116 or image quality issues related to lighting, foreign objects, etc. Product images 160 may be captured in real-time to enable a technician or caregiver with adjusting target pharmaceutical 118 to capture improved image data. In some configurations, both types of product images (product reference data and captured image data) may be displayed in product images 160.

Feature indicators may present the results of various analyses performed on the product. For example, multimodal model 138 and its constituent classifier models for each sensor type may extract feature sets based on the respective sensor data. Visual features 166 may include key visual characteristics identified by the system, such as pill size, pill shape, and pill color as identified from the image data. Spectrometry features 168 may display a spectrogram for the product based on the spectrometer data. Olfactory features 170 may show data related to the product's scent profile, such as a histogram profile of the VOCs that olfactory sensor 124 is configured to measure, based on olfactory data. Audio features 172 may present information derived from acoustic analysis, such as an acoustic response graph for a range of vibration frequencies and an indicator for a resonance frequency, if detected, based on the audio data. In some configurations, features determined by multimodal model 138 may be displayed along reference features based on pharmaceutical type 152 and/or may include classification confidence values from the sensor-specific classifiers used in multimodal model 138.

Recognition indicators may display the final analysis results to the user. Recognized type 180 may display the system's determination of the pharmaceutical product type. For successful authentication, recognized type 180 should match pharmaceutical type 152. In some configurations, recognized type 180 may include a visual indication of whether the types match, such as coloring recognized type green for high confidence matches, red for non-matches, and yellow for low confidence matches. Recognized count 182 may show the number of units identified in receptacle 116. In some configurations, receptacle 116 may be configured to accept a batch of pills and the image data analysis may include a pill counting model, such as an edge detector and corresponding counting logic, to determine the number of pills (and/or whether multiple pill types, broken pills, or other anomalies are detected). Recognition confidence 184 may indicate the system's level of certainty in its analysis results. For example, recognition confidence 184 may be the confidence value from multimodal model 138 based on the collective results of the sensor-specific classifier models. The recognition indicators may assist the user in determining whether target pharmaceutical 118 has been authenticated by system 100 and/or may trigger additional automated processing to prevent a mismatch from being administered or dispensed.

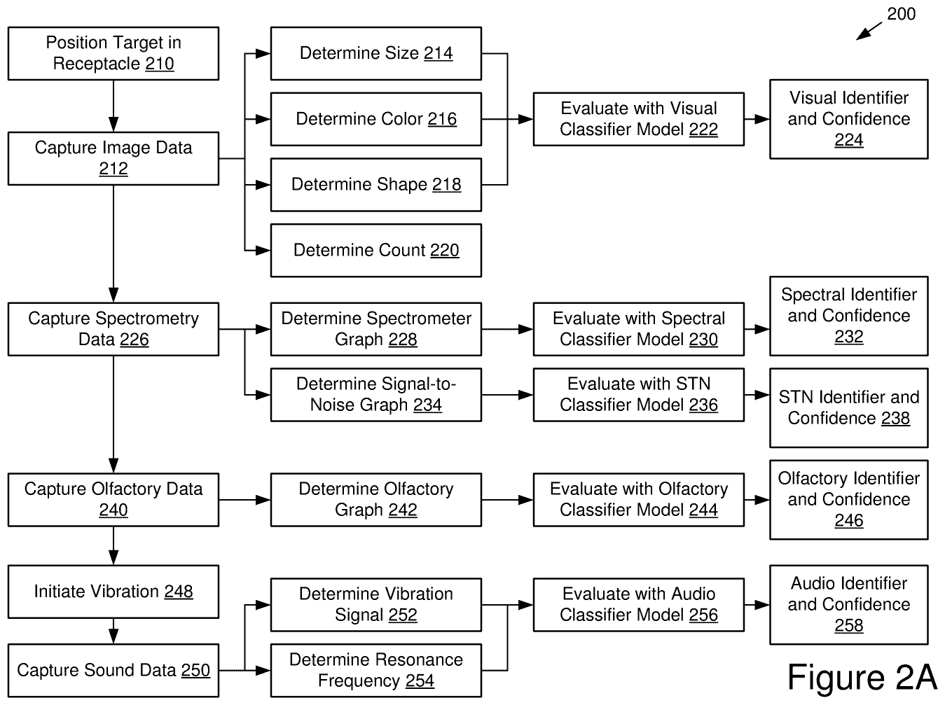

FIG. 2A illustrates a flowchart of an example method 200 for multimodal analysis of a target pharmaceutical. Method 200 may be executed by a pharmaceutical analysis system, such as system 100 shown in FIG. 1 and/or multimodal analysis engine 410 in FIG. 4. In a first stage, shown in FIG. 2A, method 200 may result in the generation of multiple identifiers and confidence levels for the target pharmaceutical based on machine learning classifier analysis of different sensor data sets. Method 200 may involve capturing and analyzing various types of sensor data, including visual, spectral, olfactory, and audio data, to comprehensively identify and authenticate a pharmaceutical product. FIG. 2B illustrates a final stage of method 200 in block 280 for providing multimodal analysis to generate an aggregate recognized type and corresponding confidence value for recognizing or authenticating the target pharmaceutical.

At block 210, the target pharmaceutical may be positioned in a receptacle. For example, a technician may place a pill in receptacle 116 of the pharmaceutical analysis system 100.

At block 212, image data may be captured. For example, camera 120 may take one or more high-resolution photographs of the pill in receptacle 116 to generate corresponding image data.

At block 214, the size may be determined. For example, image processor 416 may analyze the captured images to measure the dimensions of the pill. In some configurations, receptacle 116 may include visual size reference marks that may be used by a size model to determine the dimensions of one or more pills in receptacle 116.

At block 216, the color may be determined. For example, image processor 416 may analyze the captured images to identify the color or color pattern of the pill. In some configurations, receptacle 116 may be configured with lighting that provides a predetermined light intensity and color temperature to assist a color classifier model in consistently returning accurate color classifications.

At block 218, the shape may be determined. For example, image processor 416 may analyze the captured images to recognize the geometric shape of the pill. In some configurations, features from an edge detector may be applied to a pill shape classifier trained for a known set of pill shapes associated with the pharmaceuticals supported by the system.

At block 220, the count may be determined. For example, image processor 416 may analyze the captured images to count the number of pills present in receptacle 116. In some configurations, the same edge detection features used for shape classification may be used in a pill counting model to determine a number of pills from the image data.

At block 222, a visual classifier model may be used to evaluate the visual features. For example, image processor 416 may apply a machine learning classifier model trained on visual characteristics of known pharmaceuticals to the extracted set of visual features from blocks 214, 216, and 218. As shown in FIG. 2B, the visual classifier model may be based on baseline identification reference data in baseline identification reference library 260. For example, visual identifier reference 262 may include visual feature sets mapped to known pharmaceutical products to allow the visual classifier model at block 222 to match the collected visual data and corresponding features to at least one pharmaceutical type.

At block 224, a visual identifier and confidence may be generated. For example, image processor 416 may output a probable pharmaceutical type and a confidence score based on the visual feature analysis and classification.

At block 226, spectrometry data may be captured. For example, spectrometer 122 may collect spectral data from the pill in the receptacle 116. In some configurations, a laser may be actuated to reflect a beam off of the pill and the resulting electromagnetic reflection may be collected by an infrared sensor in spectrometer 122 to generate spectral data.

At block 228, a spectrometer graph may be determined. For example, spectral processor 418 may generate a graph representing the spectral signature of the pill. For example, a spectral graph of intensities versus wavelengths may be assembled from the response of the IR sensor to the reflected laser.

At block 230, a spectral classifier model may be used to evaluate the spectrometer graph. For example, spectral processor 418 may apply a machine learning classifier model trained on spectral signatures of known pharmaceuticals to the generated spectrometer graph to determine a closest matching spectral signature. As shown in FIG. 2B, the spectrometer classifier model may be based on baseline identification reference data in baseline identification reference library 260. For example, spectral identifier reference 264 may include spectral graphs or signatures mapped to known pharmaceutical products to allow the spectrometer classifier model at block 230 to match the collected spectral data and corresponding features to at least one pharmaceutical type.

At block 232, a spectral identifier and confidence may be generated. For example, spectral processor 418 may output a probable pharmaceutical type and a confidence score based on the spectral classifier model.

At block 234, a signal-to-noise (STN) graph may be determined. For example, the spectral processor 418 may generate a graph representing the signal-to-noise ratio of the spectral data. In some configurations, the signal-to-noise graph may map spectral peaks attributable to specific materials to spectral energy that does not match spectral signatures.

At block 236, an STN classifier model may be used to evaluate the signal-to-noise graph. For example, spectral processor 418 may apply a machine learning classifier model trained on signal-to-noise characteristics of known pharmaceuticals to the generated signal-to-noise graph to determine a closest matching SNR signature. As shown in FIG. 2B, the STN classifier model may be based on baseline identification reference data in baseline identification reference library 260. For example, STN identifier reference 266 may include signal-to-noise signatures mapped to known pharmaceutical products to allow the STN classifier model at block 236 to match the collected spectral data and corresponding features to at least one pharmaceutical type.

At block 238, an STN identifier and confidence may be generated. For example, spectral processor 418 may output a probable pharmaceutical type and a confidence score based on the signal-to-noise classifier model.

At block 240, olfactory data may be captured. For example, olfactory sensor 124 may collect chemical signature data from the air around the pill in receptacle 116. In some configurations, olfactory sensor 124 may generate signal data from the reactions of small concentrations of VOCs in receptacle 116 with different cells of a chemical sensor array.

At block 242, an olfactory graph may be determined. For example, olfactory processor 420 may generate a graph representing the chemical signature of the pill. In some configurations, olfactory processor 420 may map the signal values from the various cell positions in the sensor array to the corresponding VOCs to represent different concentrations of that VOC.

At block 244, an olfactory classifier model may be used to evaluate the olfactory graph. For example, olfactory processor 420 may apply a machine learning classifier model trained on chemical signatures of known pharmaceuticals to the generated olfactory graph. As shown in FIG. 2B, the olfactory classifier model may be based on baseline identification reference data in baseline identification reference library 260. For example, olfactory identifier reference 268 may include olfactory graphs mapped to known pharmaceutical products to allow the olfactory classifier model at block 244 to match the collected olfactory data and corresponding features to at least one pharmaceutical type.

At block 246, an olfactory identifier and confidence may be generated. For example, olfactory processor 420 may output a probable drug type and a confidence score based on the olfactory classifier model.

At block 248, vibration may be initiated. For example, vibration motor 128 may be activated by motor controller 140 to induce vibrations in the pill within receptacle 116. In some configurations, motor controller 140 may initiate a vibration pulse at a selected frequency and/or a frequency sweep across a range of frequencies with a known step and timing.

At block 250, sound data may be captured. For example, audio sensor 126 may record the acoustic response of the pill to the induced vibrations. In some configurations, the acoustic response data may include a range of audio frequencies and corresponding amplitudes.

At block 252, a vibration signal may be determined. For example, audio processor 422 may process the recorded sound data to map the audio frequencies and amplitudes to a time-based vibration signal of the pill. In some configurations, the vibration signal may correspond to a vibration pulse and/or frequency sweep induced by the motor and may be mapped to corresponding timing data for the applied vibrations.

At block 254, a resonance frequency may be determined. For example, audio processor 422 may analyze the frequency spectrum of the vibration signal to identify the resonance frequency of the pill. In some configurations, a resonance frequency may be determined from an amplitude response at a particular frequency that exceeds a resonance threshold corresponding to a non-linear response of the pill at that frequency.

At block 256, an audio classifier model may be used to evaluate the audio features. For example, audio processor 422 may apply a machine learning classifier model trained on acoustic characteristics of known pharmaceuticals to the extracted audio features. In some configurations, the acoustic characteristics may include both example vibration signals and corresponding resonance frequencies, where determined. As shown in FIG. 2B, the audio classifier model may be based on baseline identification reference data in baseline identification reference library 260. For example, audio identifier reference 270 may include audio signal graphs mapped to known pharmaceutical products to allow the audio classifier model at block 256 to match the collected audio data and corresponding features to at least one pharmaceutical type.

At block 258, an audio identifier and confidence may be generated. For example, audio processor 422 may output a probable drug type and a confidence score based on the audio classifier model.

In FIG. 2B, at block 280, a multimodal confidence value may be calculated for a consensus recognized type. For example, multimodal model 138 and/or recognition logic 424 may use the pharmaceutical identifiers and corresponding confidence values from each of the sensor classifiers to determine the overall recognized type and a corresponding degree of confidence. In some configurations, the determined visual identifier and confidence from the visual classifier model, the spectral identifier and confidence from the spectrometer classifier model, the STN identifier and confidence from the STN classifier model, the olfactory identifier and confidence from the olfactory classifier model, and the audio identifier and confidence from the audio classifier model may be input to a multimodal model based on a statistical or machine learning model. For example, an averaging model may be used to calculate recognized confidence 292, such as a simple average confidence value 282 based on adding the respective sensor confidence values and dividing by the number of values summed or weighted average confidence values 284 based on adding a weighting factor to each input confidence value. The weighting factors for the various sensor confidence terms may be determined through a machine learning algorithm and, in some cases, may include different weighting factors based on recognized type 290. For example, drug A may have a more unique reference profile for one or more sensor factors and the confidence value corresponding to that sensor's classifier may be given a greater weight value to reflect the greater correlation. In some configurations, rules-based confidence logic 286 may be used for mapping the input identifiers and confidence values to corresponding recognized types and recognized confidence (which may be based on selective averaging or weighted averaging). For example, a set of logical rules may form a transfer function for determining whether consensus may be formed from the set of inputs and, if so, how the confidence values can most accurately be combined based on the identifications made and their internal consistencies. In some configurations, rules-based confidence logic 286 may include rules for preprocessing the received classifier outputs to eliminate edge cases and/or identify error states unlikely to result in a reliable outcome. For example, rules-based confidence logic 286 may start with a simple consensus or voting rule set based on the classifier identifiers and their ability to identify a single recognized type. Consensus below a certain agreement threshold may result in an error and not be processed through a confidence model. Due to the size and complexity of this possible rule set, particularly as the number of pharmaceutical types and sensor feature sets increases, a multimodal model based on a trained multimodal classifier 288 may be used.

In some examples, trained multimodal classifier 288 may be a final classifier model that receives data from the sensor classifier models to determine recognized type 290 and recognized confidence 292. The classifier model used may include a neural network model with input nodes corresponding to a pharmaceutical type and confidence value from each sensor classifier. In other examples, one or more feature sets corresponding to each sensor classifier may be included as input nodes, resulting in a more complex and computationally intensive multimodal classifier model. The multimodal classifier model may be trained based on a plurality of example feature sets and/or identifier/confidence pairs previously classified according to known pharmaceutical types. In some configurations, baseline identification reference library 260 may be assembled based on sets of reference feature sets for each type of sensor data and this library may, in turn, be used for the training of trained multimodal classifier 288. In some configurations, a number of trained multimodal classifiers may be trained and instantiated in the system for different recognized types or classes of recognized types. More specific multimodal models may be selected for use based on the sensor classifier outputs and rules-based confidence logic 286. These more specific multimodal models may be trained to focus on closely related drugs, including generics versus name brands or different dosages of the same medication, and provide greater precision for accurately authenticating pharmaceuticals within that class or group. Each of these multimodal models may be configured to generate a recognized type with the desired level of specificity and yield an overall recognized confidence value to support the determination.

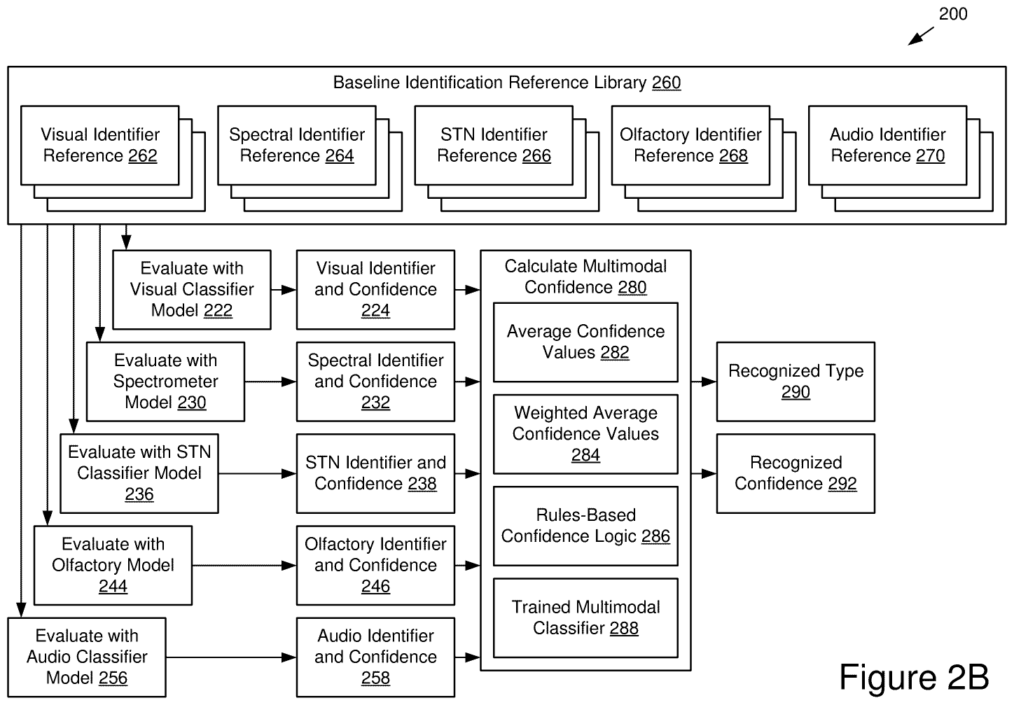

FIG. 3 illustrates a flowchart of an example method 300 for training multiple classifier models for drug recognition. Method 300 may be executed by a pharmaceutical analysis system, such as system 100 shown in FIG. 1, or a dedicated training system. For example, a computing system configured for training machine learning models may be employed to train the set of classifier models for each sensor mode and then the model parameters may be loaded into system 100 or multimodal analysis engine 410 to deploy and use those models for pharmaceutical recognition and/or authentication. Method 300 may result in the creation of trained classifier models for visual, audio, spectral, and olfactory data analysis that contribute to an overall recognition and confidence through a multimodal model. Method 300 may involve capturing various types of data from reference drug samples, extracting relevant features, and training separate classifier models for each sensor type and corresponding mode of classification, sometimes referred to as a sensor mode. For example, a camera that gathers visual data may support a number of classifier modes for different visual aspects of the target pharmaceutical, such as shape, size, color, and count. In some configurations, a model topology and training framework may be determined for each sensory mode at 302, followed by training each model sequentially at 304.

At block 310, the model type for each sensor mode may be determined. For example, the training system may select appropriate machine learning model architectures for visual, audio, spectral, and olfactory classifiers, such as neural networks having a particular topology (nodes, layers, initial path weights, and functions) compatible with using a set of input features from the corresponding sensor data to generate one or more classifications and corresponding confidence values. In some configurations, each model type may include a classifier model selected from a single class or multi-class classifier depending on the number of classification outcomes for the model.

At block 312, the learning mode may be determined. For example, the system may choose between supervised, unsupervised, or reinforcement learning approaches for training the classifiers. The learning mode may be determined by the level of pre-classification available for training data sets for the pharmaceutical types and sensor modes and may determine the learning function employed for iterating the model training.

At block 314, the training data for each sensor mode may be determined. For example, the system may compile datasets of previously collected and labeled sensor data for known drug samples, such as visual, audio, spectrometry, and olfactory data sets for different pharmaceuticals and sensor feature sets.

At block 316, feature sets for each sensor mode may be extracted. For example, the system may process the raw sensor data to identify and isolate relevant features for each sensor data set and mode of analysis. For example, each image may be preprocessed to extract edge vectors, color features, size references, and other visual data features that may be used by the respective classifier models.

At block 320, the drug to be recognized may be determined for training purposes. For example, the system may select a specific pharmaceutical product from a database of drugs requiring classifier models. A pharmaceutical type for training purposes may include a drug name, manufacturer, and dosing. All sensor data generated through blocks 322-334 may be tagged with the pharmaceutical type and stored to a baseline identification reference library for use in training, retraining, and recognition/authentication processing.

At block 322, a reference sample of the drug may be prepared. For example, a technician may obtain a verified sample of the selected drug and place it in a receptacle of the system. The configuration of the receptacle may match the configuration of the receptacle used by the field multimodal analysis system. Training may include multiple trials with the same sample and multiple samples to provide a number of training iterations with varied samples and conditions to improve the robustness of the resulting models.

At block 324, image data may be captured. For example, a camera may capture at least one high-resolution digital image of the reference drug sample. In some configurations, multiple images with varying angles and lighting conditions may be captured for the training data set.

At block 326, vibration may be initiated. For example, a vibration motor may be activated to induce controlled vibrations in the reference drug sample at a range of vibration frequencies. The vibration motor may be controlled to provide a known vibration pattern and timing calibrated to match the patterns used by the field multimodal analysis systems.

At block 328, sound data may be captured. For example, the audio sensor 126 may record the acoustic response of the reference drug sample to the induced vibrations. Multiple trials of at least one vibration pattern used by the field device may generate multiple sets of audio data for the training data set.

At block 330, the sample may be optionally pulverized. For example, a grinding mechanism may crush the reference drug sample to prepare it for spectral and olfactory analysis. In some configurations, such as batch substance control, destructive testing of a limited number of samples may be acceptable and provide stronger olfactory and spectrometry signatures. For such applications, comparable reference data may be used for training. Even if destructive testing will not be used in the field, such as for dispensing prescriptions and administering medications to patients, including reference data from pulverized samples may increase the accuracy of the classifier models by biasing the reference data toward the unique signature of a particular pharmaceutical composition.

At block 332, spectrometry data may be captured. For example, a spectrometer may collect spectral data from the intact and/or pulverized reference drug sample. Multiple trials of capturing spectral data with different samples and sample positions may be used to generate the reference data for training.

At block 334, olfactory data may be captured. For example, an olfactory sensor may collect chemical signature data from the air around the intact or pulverized reference drug sample. Multiple trials of capturing olfactory data with different samples and sample positions may be used to generate the reference data for training. In some configurations, the olfactory sensor may be reset after each sampling to clear the VOCs from the prior sampling.

At block 340, the visual feature set may be extracted. For example, an image processor may analyze the captured images to identify key visual features related to size, shape, color, and markings. One or more image processing algorithms, such as edge detectors, object detectors, color graphs, and corresponding embedding models may determine a set of image features, generally expressed as feature vectors, for input into classifier training.

At block 342, the audio feature set may be extracted. For example, an audio processor may analyze the recorded sound data to extract features such as frequency response and resonance patterns. Audio processors may map recorded sound over time and frequency to the vibration frequencies and timing used to stimulate the audio response. The resulting feature sets may include vector or matrix representations of amplitude and/or frequency data and may be filtered and/or statistically analyzed for particular response characteristics relevant to the audio signatures of pharmaceutical products.

At block 344, the spectral feature set may be extracted. For example, a spectral processor may analyze spectrometry data to identify characteristic peaks and patterns in the spectral signature. Spectral processors may map spectral data in the frequency spectrum for the reflected laser wavelengths by their measured intensities. The resulting feature sets may include vector or array representations of intensity values at each wavelength and may be filtered and/or statistically analyzed for particular response characteristics, such as peaks/intensities exceeding a relevance threshold, to represent the spectral signatures of pharmaceutical products.

At block 346, the olfactory feature set may be extracted. For example, an olfactory processor may analyze the chemical sensor data to identify unique patterns of volatile organic compounds. Olfactory processors may map olfactory data across different detected chemicals based on the amplitude of the chemical detector signals. The resulting feature sets may include vector or matrix representations of amplitude values for each chemical compound and may be filtered and/or statistically analyzed for particular response characteristics, such as amplitudes exceeding a relevance threshold, to represent the olfactory signatures of pharmaceutical products.

At block 350, the visual classifier model for the drug may be trained. For example, the system may use the extracted visual features and labeled training data to train a convolutional neural network for visual drug recognition. Training may include iterative processing of multiple reference samples in the visual training data set. In some configurations, a plurality of visual classifier models may be trained for different visual characteristics, such as size, shape, color, and markings, and/or a visual classifier model that maps features corresponding to the different visual characteristics to the drug to be recognized may be trained.

At block 352, the audio classifier model for the drug may be trained. For example, the system may use the extracted audio features and labeled training data to train a recurrent neural network for acoustic drug recognition. Training may include iterative processing of multiple reference samples in an audio training set. In some configurations, a plurality of audio classifier models may be trained for different audio characteristics, such as audio response signatures at different frequencies of vibration and/or resonance frequency from an audio response spectrum, and/or an audio classifier model that maps features corresponding to different audio characteristics to the drug to be recognized.

At block 354, the spectral classifier for the drug may be trained. For example, the system may use the extracted spectral features and labeled training data to train a support vector machine for spectral drug recognition. Training may include iterative processing of multiple reference samples in a spectral training set. In some configurations, a plurality of spectral classifier models may be trained for different laser and sensor array configurations and target wavelengths, such as spectral response signatures at different wavelength ranges, and/or a spectral classifier model that maps features corresponding to different spectral characteristics to the drug to be recognized.

At block 356, the olfactory classifier for the drug may be trained. For example, the system may use the extracted olfactory features and labeled training data to train a random forest classifier for chemical signature recognition. Training may include iterative processing of multiple reference samples in an olfactory training set. In some configurations, a plurality of olfactory classifier models may be trained for different chemical sensor array configurations and target VOCs, such as a sensor system combining the results of multiple chemical sensor arrays selected for different groups of compounds common in pharmaceutical products of interest, and/or an olfactory classifier model that maps features corresponding to different olfactory characteristics to the drug to be recognized.

At blocks 360, 362, 364, and 366, the visual, audio, spectral, and olfactory model metrics may be evaluated against corresponding thresholds. For example, the system may compare the accuracy, precision, and recall of the trained classifiers against predetermined performance benchmarks to determine whether each one meets production-level reliability for the drug to be recognized. If not, additional reference data may be generated and/or added to the training data set for additional iterations of training or retraining and/or one or more aspects of the model ontology and training framework may be reconsidered by repeating one or more of the blocks at 302.

At blocks 370, 372, 374, and 376 the model parameters for the visual, audio, spectral, and olfactory classifier models for each drug type to be recognized may be determined. For example, the system may determine the parameter set for each classifier model and corresponding drug type to generate a set of field classifier models that may be stored in a model library and used by field multimodal analysis systems. The model library or a subset of the model library may then be deployed to multimodal analysis systems at configuration or dynamically as needed. For example, a multimodal analysis engine or multimodal device may be loaded with model sets selected for the pharmaceutical types they are configured to recognize or authenticate, based on their application or using an on-demand mode where the authentication target is used to index and retrieve the set of models for that pharmaceutical type.

As noted elsewhere, the set of classifier models may further include a multimodal classifier model configured to take the pharmaceutical types, confidence values, and/or other features or outputs from the visual, audio, spectral, and olfactory classifier models and process them to provide an aggregate pharmaceutical type decision and corresponding overall classification confidence value. The multimodal classifier model may be configured and trained similarly to the set of constituent classifier models and based on their outputs as training data. For example, the system may configure the multimodal classifier model with a model ontology and training framework according to the blocks at 302. The extracted feature set may correspond to the outputs of the constituent classifiers and the training process may use the set of models qualified at blocks 370-376 to reprocess their respective training data sets to provide the iterative training of the multimodal classifier model. The iterative multimodal classifier model may be evaluated using similar metrics and thresholds and result in a set of multimodal classifier models for different drug types that may be added to the model library.

FIG. 4 shows an example implementation of system 400 for virtual verification of prescription products using multimodal analysis to automate and streamline authentication by a pharmacist that does not have physical access to the target prescription product being dispensed. As shown in FIG. 4, a retail pharmacy system including remote dispensing site 440 and a supervising pharmacy 470, and an enterprise pharmacy data system 432 may be configured to interact with a multimodal analysis engine 410 to enable virtual verification of prescription product in a pharmacy prescription fill workflow. The remote dispensing site 440 and supervising pharmacy 470 may be at separate physical sites or may be co-located within the same physical site but physically separated (e.g., at different workstations, in different buildings, in different rooms, etc.). Multimodal analysis engine 410 may be embodied in a combination of software modules supporting the pharmacy dispensing application 446 and verification workflow 448 at the remote dispensing site, locally on the pharmacy computing device 444, integrated in multimodal device 452, and/or served over a network from the enterprise pharmacy data system. For example, some data and functions may be stored in local data on the pharmacy computing device and/or connected multimodal device, while other data and functions may be accessed through an application programming interface (API) in the enterprise pharmacy data system. U.S. Pat. No. 12,027,247 is hereby incorporated by reference in its entirety except insofar as any subject matter that is contrary to the explicit disclosure herein is not incorporated.

Multimodal analysis engine 410 may be configured to receive and analyze sensor data from multimodal device 452 and generate drug recognition and confidence data for display through multimodal display 478. The engine may include hardware components such as specialized processors, field programmable gate arrays (FPGAs), systems on a chip (SOCs) or application specific integrated circuits (ASICs), and software components such as machine learning libraries and data processing algorithms. In some configurations, multimodal analysis engine 410 may be embodied in an ASIC comprised of one or more neural network circuits and corresponding data registers for input, output, and intermediate data for each machine learning model in the engine. For example, image processor 416, spectral processor 418, olfactory processor 420, audio processor 422, and recognition logic 424 may each be embodied in one or more corresponding neural network circuits and pass intermediate results (such as recognition types and confidence values) among the processors via a configuration of input and output data registers. Similarly, prescription interface 412 and collected data interface 414 may include input data registers and corresponding preprocessing logic and filters for sampling and directing sensor data to the corresponding input registers for the various processor circuits. Each neural network circuit may include parameter registers configured to receive trained parameter values (e.g., according to method 300), such as weighting values for the various nodes and layers of each neural network based on the neural network topology selected for that model. In some configurations, count logic 426 and anomaly logic 428 may comprise additional neural network circuits that are incorporated in or interface with the multimodal analysis ASIC. In some configurations, the multimodal analysis ASIC may be integrated in or interface with another computing system, such as enterprise pharmacy computing system 432, pharmacy computing device 444, and/or computing device 474.

Prescription interface 412 may include logic, data structures, and/or APIs for accessing prescription data for a prescription being filled at remote dispensing site 440. For example, prescription interface 412 may receive prescription data, including patient data for the patient prescribed the drug, pharmaceutical type (drug name and dosage), and pharmaceutical count (number of units to be filled), from dispensing application 446 and/or pharmacy services 434. Collected data interface 414 may receive and preprocess raw sensor data from the multimodal device 452, such as image data, spectral data, olfactory data, and audio data. For example, after the target pharmaceutical for the prescription has been placed in receptacle 454, multimodal device 452 may initiate or be controlled to collect data about the sample using its various sensor modes and provide that data through a data transfer interface to multimodal analysis engine 410.

Image processor 416 may extract visual features from image data from camera 456, such as pill shape, size, and color. For example, image processor 416 may be configured to execute the visual data portion of method 200. Spectral processor 418 may analyze spectrometer data from spectrometer 458 to identify characteristic spectral signatures of pharmaceuticals. For example, spectral processor 418 may be configured to execute the spectral data portion of method 200. Olfactory processor 420 may process chemical sensor data from olfactory sensor 460 to detect unique volatile organic compound patterns. For example, olfactory processor 420 may be configured to execute the olfactory data portion of method 200. Audio processor 422 may analyze acoustic data from audio sensor 462 to determine resonance frequencies and other auditory properties of pills. Recognition logic 424 may apply logical, statistical, and/or trained machine learning models to the processed sensor data to identify the pharmaceutical type and generate confidence scores. For example, recognition logic 424 may be configured to execute block 280 of method 200. In some configurations, each processor 416, 418, 420, and 422 and/or recognition logic 424 may be embodied in hardware and/or software subsystems configured for their specific models, such as one or more classifier models trained for their sensor modes. For example, each classifier may be embodied in a neural network processor with corresponding memory resources configured to efficiently execute the respective embedding extractions and classifier models based on the trained sets of model parameters generated using method 300. In some configurations, processors 416, 418, 420, and 422 and/or recognition logic 424 may utilize a model library 430 to load the model parameters for the target pharmaceutical being analyzed. For example, using the prescription type from the prescription data received through prescription interface 412 as an index value, multimodal analysis engine 410 may select the corresponding set of models for the sensor modes and multimodal recognition model trained for that prescription type.

In some configurations, multimodal analysis engine 410 may include additional analytics to support virtual verification workflow 476. Count logic 426 may determine the number of pills present in the receptacle based on image analysis. For example, an edge detector and corresponding machine learning model for pill counting may be embodied in a corresponding processor subsystem. Anomaly logic 428 may detect any irregularities or unexpected features in the analyzed data that may indicate obstructions, error states, or compromised pharmaceuticals. For example, error conditions for each sensor mode may be configured in anomaly logic 428 to raise errors to the users and interrupt recognition processing for: broken pills, pills of different sizes, or foreign objects; poor image quality induced by motion or lighting; spectral, olfactory, or auditory amplitudes outside of normal ranges; indicators of sensor malfunction, and other anomalies or error states. In some examples, the multimodal analysis engine 410 may utilize cloud computing resources to perform computationally intensive tasks, such as running complex neural network models for drug recognition. In some examples, the engine may incorporate a federated learning system to continuously improve its models by aggregating insights from multiple remote dispensing sites while maintaining data privacy.

In some implementations, a pharmacy system may include a pharmacy computing device 444 and a multimodal device 452 including a camera 456 and a number of other sensors, such as spectrometer 458, olfactory sensor 460, and audio sensor 462. Pharmacy computing device 444 and remote computing device 474 used by the pharmacist and/or technician may similarly use a combination of local computing resources and network computing resources through network 402 for coupling with enterprise pharmacy computing system 432 and/or multimodal analysis engine 410. Multimodal device 452 may be configured to be coupled to the pharmacy computing device 444 for capturing high quality images and other sensor data for a prescription product in receptacle 454. In some implementations, the captured data from multimodal device 452 may be captured and adjusted (e.g., white balance, noise reduction, etc.) using the pharmacy computing device 444 and subsequently sent to multimodal analysis engine 410 for analysis. Pharmacy computing device 444 may include or access control functions of multimodal device 452 to initiate capture of images and other sensor data, as well as initiating other control functions, such as resetting sensors, adjusting lighting, or initiating vibration motor 464.

Pharmacy dispensing application 446 may control or receive data from the enterprise pharmacy data system 432 and multimodal analysis engine 410, and identify and format the relevant data for presentation to the pharmacist 104 and/or technician 102. In some implementations, verification workflow 448 may be part of the prescription fulfillment workflow in a pharmacy system 400. In some implementations, the information for presentation to the pharmacy staff may be displayed on a multimodal display 450 of the pharmacy computing device 444. There may be multiple pharmacy computing devices 444/474 configured to interact with each other and the enterprise pharmacy computing system 432. For example, it may be that some retail pharmacies act as supervising pharmacies/locations 470 and house a pharmacist 472 to oversee and verify the prescription workflow of a technician 442 in a telepharmacy, other retail pharmacy location, or other remote location. Data captured by multimodal device 452 and processed by multimodal analysis engine 410 may be displayed on multimodal displays 450 and 478 at the two locations. Technician 442 may use multimodal display 450 to verify accurate capture of sensor data and/or correct/repeat data capture in response to errors or anomalies detected by anomaly logic 428 and/or based on feedback from pharmacist 472 provided through virtual verification workflow 476. Pharmacist 472 may use multimodal display 478 to review the recognition and confidence indicators generated by multimodal analysis engine 410 and provide verification for dispensing the prescription and releasing it to the patient or identifying one or more conditions for denying verification and providing appropriate feedback to technician 442 through virtual verification workflow 476. In some configurations, multimodal display 478 may be configured similarly to graphical user interface 112 in FIG. 1 and/or integrate similar data fields and/or visual indicators into the existing user interface for dispensing application 446 and, more specifically, verification workflow 448 and/or virtual verification workflow 476.

In some implementations, enterprise pharmacy computing system 432 may host a number of pharmacy services 434 and a drug database 436. For example, pharmacy services 434 may include prescription reorder, prescription delivery, linkage to specific savings programs, subscription fill services, bundling additional prescriptions for refill/pickup, automating next refill, conversion to 90-day prescriptions, clinic services, flu shots, vaccines, non-prescription products, etc. Drug database 436 may include information about prescription and over-the-counter medication. In particular, drug database 436 may include proprietary or in-house databases maintained by pharmacies or drug manufacturers, commercially available databases, and/or databases operated by a government agency. Drug database 436 may be accessed using industry standard drug identifiers, such as without limitation, a generic product identifier (GPI), generic sequence number (GSN), national drug code directory (NDC), universal product code (UPC), health related item, or manufacturer. In some configurations, drug database 436 may include or link to a sensor data reference library 438 of aggregated sensor data that has been associated with specific pharmaceutical types (drug identifier, including dosage). For example, the sensor data in reference library 438 may be used for model training as described for method 300 and may receive additional sensor data from ongoing field operations to improve and expand the reference data set for future training or retraining of models.

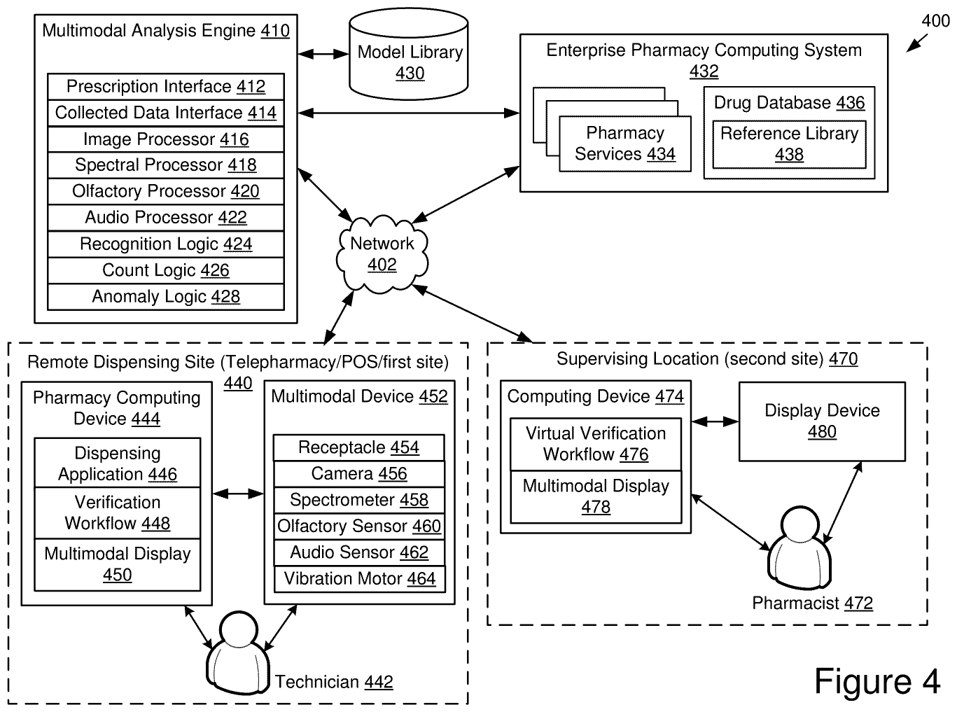

An example configuration of multimodal device 452 is shown in different views in FIGS. 5A-5C as multimodal device 500. In some implementations, multimodal device 500 uses a Counting and Imaging Tray (CAIT) 560 that provides a removable counting tray that acts as the receptacle for the prescription products to be dispensed. CAIT 560 may be used by a pharmacy staff member, such as technician 442, to both count and collect sensor data for the prescription, such as pills without needing to dump the pills from the tray into another container or tray for capturing images and other sensor data. The pharmacy staff member may:
1—Pour pills from a stock bottle of the prescription product onto a first portion or a counting level 564 during a prescription workflow;
2—Count and swipe the prescribed quantity of pills onto a second portion of imaging level 566;
3—Pour the remaining amount on the counting level back into the stock bottle via a spout or chute at one of the corners of counting level 564;
4—Slide CAIT 560 into multimodal device 500 through an opening 562 in a side wall (e.g., side wall 512) to capture sensor data for the prescribed prescription type and quantity; and
5——Pour the contents into a vial or bottle via another spout or chute at one of the corners of imaging level 566.

CAIT 560 may be particularly advantageous because of the different spouts or chutes for inputting and dispensing the prescription, the different layers at different heights for counting and imaging, and the walls around each layer of the CAIT that are sloped or angled to bias or direct the pills towards the next area of processing from input spout or chute to output spout or chute. In one aspect, CAIT may be further configured with at least one slope within the second portion of the tray (e.g., imaging level 566) to bias the pills toward the field of view of the camera 530 and a target area for the other sensors. CAIT 560 may position the target pharmaceuticals in proximity to spectrometer 546, olfactory sensor 548, and audio sensor 550. For example, spectrometer 546 may be positioned relative to CAIT 560 to direct a laser at a target portion of imaging level 566 and receive reflected light in its electromagnetic spectrum sensor array. Technician 442 may be able to adjust the position of one or more pills to assure that the laser reflects off of its surface. Olfactory sensor 548 may be in close proximity to the imaging level and, once door 522 is closed, may collect VOCs primarily from the pills in CAIT 560. Audio sensor 550 may be in similar proximity to the imaging level for gathering sound data and vibration motor 552 may be configured to directly engage CAIT 560 (e.g., in contact with the underside of the imaging level) to vibrate the pills according to the vibration pattern for audio data collection and analysis.

In some configurations, a single camera 530 is illustrated for capturing high quality images of the prescription product using CAIT 560. In another aspect, a dual camera (e.g., first camera 530 and second camera 534) configuration is illustrated for capturing high quality images of the prescription product using CAIT 560, depending on whether it is positioned inside multimodal device 500 or outside multimodal device 500. Multimodal device 500 includes an enclosure 510 for housing and supporting various structures, including first camera 530 on mounting bracket 532. Enclosure 510 may include a plurality of walls including side walls 512 and 514, back wall 516, top wall 518, and base 520. These walls define an interior portion of enclosure 510 that may be further defined by a door 522 operable to enclose the interior of enclosure 510 during some operations. First camera 530 may be configured to attach to mounting bracket 532 on the top inner surface of the enclosure that positions the camera above a working surface to provide a field of view 540 over the working surface. Field of view 540 may be configured to capture the imaging level 566 of CAIT 560 when it is positioned inside enclosure 510.

Door 522 may be configured to provide access to imaging level 566 of CAIT 560 when the CAIT 560 is inserted into multimodal device 500. In some configurations, enclosure 510 may include an opening 562 defined in side wall 512 for inserting the imaging portion of CAIT 560 to dispose imaging level 566 in field of view 540. In operation, the CAIT may be inserted into the imaging device 500 through opening 562 when door 522 is open or closed. In either case, door 522 may be closed to improve the consistency of imaging and other sensor readings by multimodal device 500. Door 522 may include a handle 524 for opening and closing the door and a latch 526, such as a magnetic latch, for retaining the door in the closed position during operations. The interior of the imaging device in field of view 540 is protected from intermittent exterior lighting variations and other environmental interference by the closure of door 522. Accordingly, to provide improved and consistent lighting conditions for first camera 530 to capture images of prescription product in imaging level 566, multimodal device 500 may further include one or more interior lights 544. In one example, lights 544 are configured to illuminate the imaging portion of CAIT 560 with a known and consistent brightness, color temperature, and distribution for more consistent image quality. For example, lights 544 may be a row of light emitting diode (LED) lights with known properties and surrounding multiple sides of the inside of enclosure 510. In some configurations, lights 544 may be selectively operable to be on during image capture but turn off during other operations, such as capturing spectroscopy data.

Multimodal device 500 may include a plurality of additional sensors for capturing data from the pharmaceutical products placed in enclosure 510. As described above, these additional sensors may include a spectrometer 546, an olfactory sensor 548, and an audio sensor 550. Collection of audio data using audio sensor 550 may be further supported by vibration motor 552. Each of these sensor components may be positioned within the interior of enclosure 510, such as mounted to one or more side walls, base, or ceiling, and configured to gather their respective sensor data from pharmaceutical products positioned in imaging level 566 of CAIT 560. For example, spectrometer 546 may be positioned above imagine level 566 with sufficient clearance and angles for directing a laser at the pharmaceutical product and receiving the reflected light from that product. Olfactory sensor 548 may be positioned above the side wall of CAIT 560 but in close proximity to the pharmaceutical product to improve the capture of VOCs from the product. Audio sensor 550 and vibration motor 552 may be positioned to engage CAIT 560 and receive the audible response from vibration of the pharmaceutical product. In some configurations, one or more sensors may be mounted to CAIT 560 for gathering sensor data and may include a separate wireless interface for sending that data to control electronics 554 or directly to another system hosting the multimodal analysis engine.

Multimodal device 500 may include onboard electronics for controlling and/or providing a computer interface to the various sensor and other electrical assemblies of multimodal device 500. For example, control electronics 554 may include one or more circuit assemblies comprised of control circuits for video cameras 530 and 534, lights 544, spectrometer 546, olfactory sensor 548, audio sensor 550, and vibration motor 552. Each control circuit may include or interface with hardware and/or software control functions for initializing, initiating data collection operations, and receiving data from those sensors. In some configurations, control electronics 554 may be paired with control interface 556 to aggregate control functions and data streams from the sensors and other components to provide a unified peripheral interface to a computer system or network hosting the multimodal analysis engine. For example, control electronics 554 may include a processor and memory for selectively controlling and receiving data from the various sensors and providing control functions and sensor data through a standard wired or wireless computer interface, such as a universal serial bus (USB), peripheral component interconnect express (PCIe), Ethernet, WiFi, Bluetooth, or similar computer component interface. In other configurations, control electronics 554 and control interface 556 may be distributed among the sensor components and one or more sensors may incorporate their own control electronics and interface for direct communication with a data collection system or multimodal analysis engine. For example, one or more sensors may include onboard wired or wireless communication protocols that can be directly connected to a network or host computer device.

In some configurations, multimodal device 500 may include second camera 534 coupled to an exterior surface of enclosure 510. Second camera 534 may be utilized when imaging prescription product needing a field of view 542 greater than field of view 540 within the enclosure. For example, if a tray including prescription product is too large to be received within the enclosure 510 or the prescription product is too large for the tray, then external second camera 534 may be utilized. External camera 534 may be mounted to a positioning arm 536 for positioning field of view 542 outside of enclosure 510. In some configurations, positioning arm 536 may be removably mounted to brackets 538 on an exterior surface of enclosure 510 or include a freestanding base to be placed adjacent to enclosure 510. In other implementations, second camera 534 may also be used for additional capacity by multimodal device 500 and/or related imaging functions, such as imaging product packaging, prescription labels, instruction sheets, and other prescription materials other than the pills themselves.

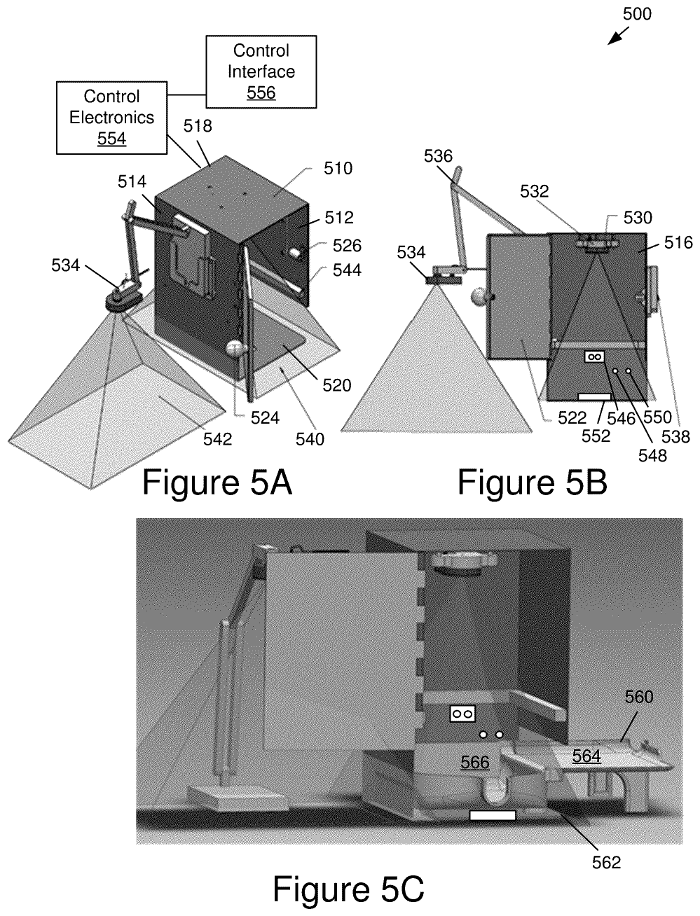

FIG. 6 is a block diagram of an example configuration of a pharmaceutical analysis system 600 and method 602 for using the system. One architecture for the multimodal device and data processing system described above may include a portable multimodal device 620 to provide sensor data acquisition at remote locations and uses a connected computing device 610 to provide multimodal data display 612. For example, multimodal device 620 may include a small receptable 622 sized for a small number or even a single pill. In some configurations, multimodal device 620 may be small enough to be carried and/or worn on a wrist strap, belt clip, ergonomic handle, or similar device for easy access and use during home health visits of a caregiver, nursing rounds, or similar patient administration activities. Receptacle 622 may include a housing or enclosure for receiving a target pharmaceutical, such as a pill, and may include a door that may be opened for receiving the sample and closed during sensor operations. As described for larger multimodal devices, such as multimodal device 500, multimodal device 620 may include a sensor package 624 comprising a plurality of sensors that may include at least one imaging sensor (camera), at least one spectroscope, at least one olfactory sensor, and at least one audio sensor disposed in or adjacent to receptacle 622 to capture multiple modes of sensor data from a target pharmaceutical placed in the receptacle.

Multimodal device 620 may include control electronics 626 for sensor package 624 that include a set of onboard control circuits for the sensors and any other electrical components, such as a vibration motor and lights in receptacle 622. For example, control electronics 626 may include control electronics for each sensor to support initialization, activation, and receipt of analog or digital data signals for each sensor mode, at least one processor, memory, and firmware for managing control signals to the various sensors and receive their data, and an interface circuit and corresponding port or antenna for communicating control and/or data signals from/to computing device 610. In some configurations, control electronics 626 may be embodied on a printed circuit board assembly (PCBA) that provides conductive traces among the varies sensor control, processor, memory, and specialized electronics attached to the board. In some configurations, the communication interface to computing device 610 may include a wireless interface 628 that may be integrated with or in communication with control electronics 626 and/or sensor package 624. For example, wireless interface 628 may include hardware and software compliant with Wi-Fi, Bluetooth, or similar wireless communication technology for establishing communication between multimodal device 620 and computing device 610. In some configurations, multimodal device 620 may be a special-purpose computing device with components similar to computing device 800, sensor package 624 including one or more input devices. In some configurations, multimodal device 620 may include one or more user interface components, such as input buttons and/or output features such as configurations of indicator lights or an LED screen. For example, multimodal device 620 may include indicator lights or icons on an LED screen corresponding to the recognized confidence or other output from the larger and more complete multimodal data display on computing device 610.

Computing device 610 may include a general purpose or specially designed computing device for receiving and displaying multimodal sensor data from multimodal device 620. In some configurations, computing device 610 may include a mobile computing device, such as a smartphone or tablet, of a caregiver or nurse configured with an application for interfacing with multimodal device 620 and displaying multimodal data display 612. In other configurations, a desktop computer, laptop, or specialized display appliance, such as the computing systems located in hospital rooms or on movable carts in healthcare facilities and nursing homes, may be configured as computing device 610. Computing device 610 may be configured to use its memory and processor(s) to execute an application comprising instructions for controlling multimodal device 620, receiving multimodal sensor data from multimodal device 620, and displaying multimodal data display 612, such as was described above for FIG. 1. Computing device 610 may include a wireless multimodal device interface 614 configured for communication with multimodal device 620, such as a Bluetooth or Wi-Fi interface. In some configurations, computing device 610 and multimodal device 620 may be configured for wired communication using a direct interface standard, such as USB or PCIe, or using network communication over Ethernet and/or another network protocol. In some configurations, computing device 610 may integrate the data processing functions of multimodal analysis engine 616 and use its processing and memory resources to access model and/or reference library 618 and execute the series of models that support the multimodal model for recognizing or verifying the target pharmaceutical in receptacle 622. In other configurations, computing device 610 may use network communication with another computing device hosting multimodal analysis engine 616.

System 600 may be operated according to method 602 for verifying or recognizing a target pharmaceutical. At block 630, a sample of the target pharmaceutical may be placed in the receptacle of a multimodal device. For example, receptacle 622 may receive a pill to be analyzed and the user may close the door to initiate analysis of the sample.

At block 632, a verification target or recognition search may be selected. For example, computing device 610 or multimodal device 620 may receive an input from a user or another system indicating a verification target, such as the pharmaceutical type in the prescription to be administered by the user to a patient, or indicating an unknown pharmaceutical to be identified.

At block 634, sensor data may be acquired. For example, multimodal device 620 may use sensor package 624 to acquire multiple modes of sensor data from the sample placed in the receptacle, such as image, spectral, olfactory, and audio sensor data.

At block 636, sensor data may be transmitted from the multimodal device to the computing device. For example, multimodal device 620 may transmit the captured sensor data for the sample to computing device 610 for analysis.

At block 628, multimodal analysis may be executed. For example, computing device 610 and/or another computing device in network communication with computing device 610 may execute multimodal analysis using a multimodal analysis engine.

At block 630, verification for a recognized type and confidence may be displayed on multimodal data display 612. For example, the multimodal analysis may determine the pharmaceutical type matching the sample with a corresponding confidence value and may display a verification indicator on the multimodal data display where the identified pharmaceutical type matches the verification target and/or provide the determined pharmaceutical type for a recognition search.

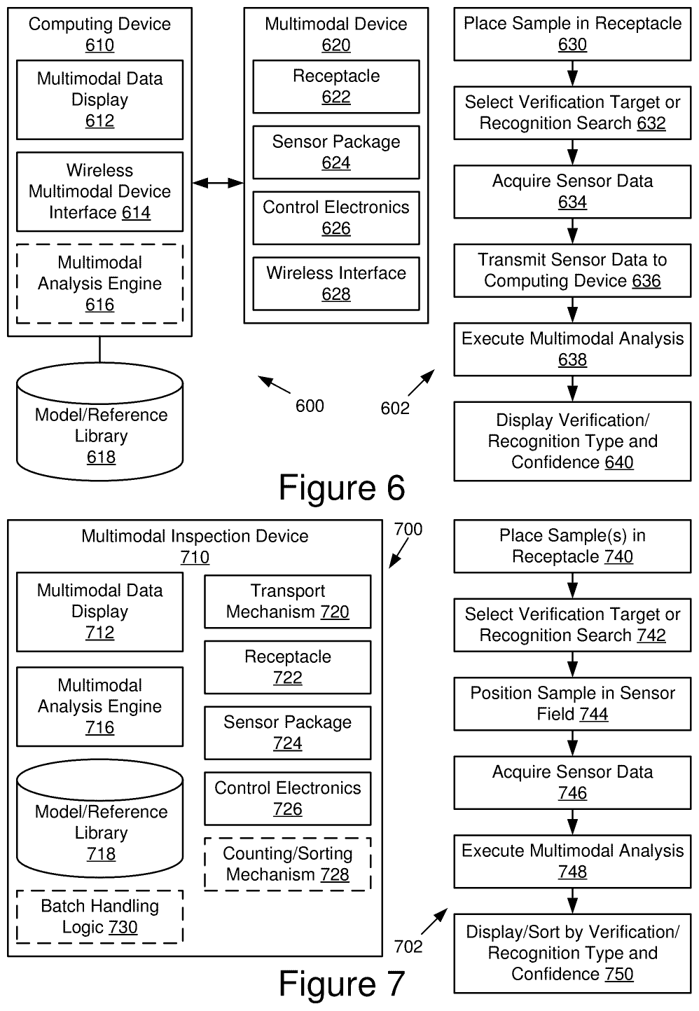

FIG. 7 is a block diagram of an example configuration of a pharmaceutical analysis system 700 and method 702 for using the system. Another architecture for the multimodal device and data processing system described above may include a multimodal inspection device 710 that integrates the multimodal device with a computing device capable of multimodal analysis to provide volume processing of samples for higher throughput applications, such as inspection, security, and manufacturing. For example, multimodal inspection device 710 may include a receptacle 722 sized and configured for batches of pills to be analyzed and paired with a transport mechanism 720 that moves the pills in and out of receptacle 722 and/or the testing area for receptacle 722. In some configurations, transport mechanism 720 may include a conveyer belt, gravity fed chute or slide, or similar mechanism for depositing target pharmaceuticals in receptacle 622 As described for other multimodal devices, such as multimodal device 500, multimodal inspection device 710 may include a sensor package 724 comprising a plurality of sensors that may include at least one imaging sensor (camera), at least one spectroscope, at least one olfactory sensor, and at least one audio sensor disposed in or adjacent to receptacle 722 and/or a testing area through which the receptacle may move to capture multiple modes of sensor data from target pharmaceuticals in the receptacle.

Multimodal inspection device 710 may include control electronics 726 for sensor package 724 that include a set of onboard control circuits for the sensors and any other electrical components, such as a vibration motor and lights in receptacle 722. For example, control electronics 726 may include control electronics for each sensor to support initialization, activation, and receipt of analog or digital data signals for each sensor mode, and at least one interface for using the processors, memories, and software of multimodal inspection device for managing control signals to the various sensors and receive their data. In some configurations, control electronics 626 may be embodied on a printed circuit board assembly (PCBA) that provides conductive traces among the varies sensor control, processor, memory, and specialized electronics attached to the board and may interface with a peripheral interface and/or motherboard of the computing system components of multimodal inspection device 710. In some configurations, individual sensors and/or other electronics (e.g., vibration motor, lights, etc.) may include their own wireless, network, and/or computer peripheral interfaces for communicating with the computing system in multimodal inspection device 710. In some configurations, multimodal inspection device 710 may include one or more user interface components in addition to a primary graphical user interface display device for multimodal data display 712, such as input buttons and/or output features such as configurations of indicator lights and/or LED screens.

Multimodal inspection device 710 may include a general purpose or specially designed computing device for receiving, processing, and displaying multimodal sensor data from sensor package 724, as well as providing control functions for sensor package 724 and other electrical components, such as motor control functions of transport mechanism 720, a vibration motor, counting/sorting mechanism 728, lights, and other components. In some configurations, multimodal inspection device 710 may incorporate one or more computing devices configured similar to computing device 800, such as a desktop computer, server, laptop, tablet, smartphone, or specialized computing appliance, such as the computing systems integrated in manufacturing, security, and/or inspection lines. Multimodal inspection device 710 may be configured to use its memory and processor(s) to execute an application comprising instructions for controlling the various system components, receiving multimodal sensor data from sensor package 724, and displaying multimodal data display 612, such as was described above for FIG. 1. In some configurations, multimodal inspection device 710 may integrate the data processing functions of multimodal analysis engine 716 and use its processing and memory resources to access model and/or reference library 718 and execute the series of models that support the multimodal model for recognizing or verifying the target pharmaceuticals in receptacle 722. In other configurations, multimodal inspection device 710 may use network communication with another computing device hosting multimodal analysis engine 716.

In some configurations, multimodal inspection device 710 may include or interface with batch handling logic 730 to coordinate analysis with the movement of transport mechanism 720 and/or receptacle 722 for sequentially analyzing batches of target pharmaceuticals. For example, batch handling logic 730 may synchronize batch identifiers with physical positioning of target pharmaceuticals based on transport mechanism 720. These batch identifiers may be used to identify and track the sensor data corresponding to the batch through the verification analysis and presentation of the analysis results through multimodal data display 712. In some configurations, multimodal inspection device 710 may include at least one counting/sorting mechanism that may be integrated into transport mechanism 720 before or after collection of sensor data. For example, a pill counter for determining a number of pills in a batch and/or pill sorter for separating pills based on size or other physical characteristics may be used to manipulate the contents of batches before or after collection of sensor data. In one example, a pill counter may determine the number of pills in a batch based on mechanical or machine vision metering of the pills and image analysis from the sensor data may be used to verify a correct count of pills appears in the resulting batch. Similarly, a mechanical pill sorter may be employed to separate a mixed collection of pills into batches of the same physical type for sensor analysis.

System 700 may be operated according to method 702 for verifying or recognizing the pharmaceutical type from the samples. At block 740, samples of the target pharmaceutical may be placed in one or more receptacles of a multimodal inspection device. For example, receptacle 722 may receive a group of pills to be analyzed.

At block 742, a verification target or recognition search may be selected. For example, multimodal inspection device 710 may receive an input from a user or another system indicating a verification target, such as the pharmaceutical type in the sample to be verified, or indicating an unknown pharmaceutical to be identified.

At block 744, the sample may be positioned in the sensor field of view by or with the receptacle. For example, receptacle 722 may be moved by transport mechanism 720, such as a conveyor belt, into the field of view or field of detection of the various sensors, which may include different positions for different sensors.

At block 746, sensor data may be acquired. For example, multimodal inspection device 710 may use sensor package 724 to acquire multiple modes of sensor data from the sample placed in the receptacle, such as image, spectral, olfactory, and audio sensor data.

At block 748, multimodal analysis may be executed. For example, multimodal inspection device 710 and/or another computing device in network communication with multimodal inspection device 710 may execute multimodal analysis using a multimodal analysis engine.

At block 750, verification for a recognized type and confidence may be displayed on multimodal data display 712. For example, the multimodal analysis may determine the pharmaceutical type matching the sample with a corresponding confidence value and may display a verification indicator on the multimodal data display where the identified pharmaceutical type matches the verification target and/or provide the determined pharmaceutical type for a recognition search. In some configurations, the verification or failure to verify the target pharmaceutical to a confidence threshold may cause multimodal inspection device 710 to mechanically separate or sort the sample to an alternative transport path and/or isolate it for further inspection, analysis, and/or seizure.

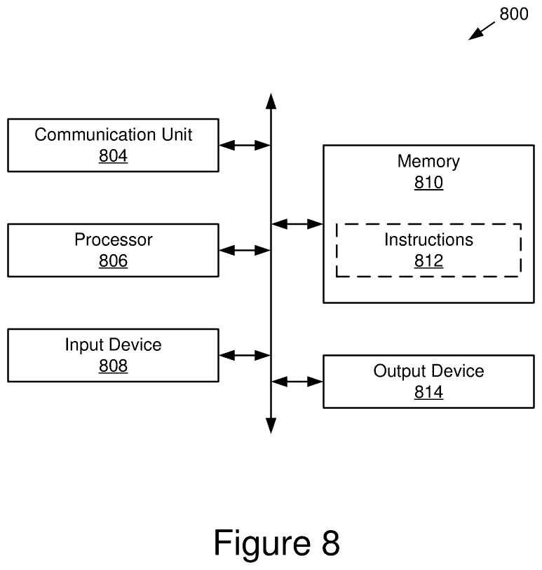

FIG. 8 is a block diagram of an example computing device 800, which may represent the computer architecture of a pharmacy computing device, multimodal devices, servers hosting enterprise pharmacy data systems, reference or model libraries, and/or the multimodal analysis engine, and/or other computing devices used in pharmaceutical analysis systems, such as systems 100, 600, and 700. As depicted, computing device 800 may include at least one processor 806, at least one memory 810, at least one communication unit 804, at least one input device 808, and at least one output device 814, which may be communicatively coupled by a bus 802. Computing device 800 depicted in FIG. 8 is provided by way of example and it should be understood that it may take other forms and include additional or fewer components without departing from the scope of the present disclosure. For instance, various components of computing device 800 may be coupled for communication using a variety of communication protocols and/or technologies including, for instance, communication buses, software communication mechanisms, computer networks, etc. While not shown, computing device 800 may include various operating systems, sensors, additional processors, and other physical configurations. Processor 806, memory 810, communication unit 804, etc., are representative of one or more of these components that may operate alone or in combination to complete their designated functions. Processor 806 may execute software instructions by performing various input, logical, and/or mathematical operations. Processor 806 may have various computing architectures to process data signals (e.g., CISC, RISC, etc.).

Processor 806 may be physical and/or virtual, and may include a single core or plurality of processing units and/or cores. In some implementations, processor 806 may be coupled to memory 810 via the bus 802 to access data and instructions 812 therefrom and store data therein. Bus 802 may couple processor 806 to the other components of the computing device 800 including, for example, memory 810, communication unit 804, input device 808, and output device 814. Memory 810 may store and provide access to data to the other components of computing device 800. Memory 810 may be included in a single computing device or a plurality of computing devices. In some configurations, memory 810 may store instructions 812 and/or data that may be executed by the processor 806. For example, memory 810 may store one or more of the multimodal analysis engines, multimodal data interfaces, dispensing applications, workflow system, pharmacy services, verification workflow, etc. and their respective components, depending on the configuration. Memory 810 is also capable of storing other instructions and data, including, for example, an operating system, hardware drivers, interface protocols, other software applications, databases, etc. Memory 810 may be coupled to bus 802 for communication with the processor 806 and the other components of computing device 800.

The memory 1510 may include a non-transitory computer-usable (e.g., readable, writeable, etc.) medium, which can be any non-transitory apparatus or device that can contain, store, communicate, propagate or transport instructions 1512, data, computer programs, software, code, routines, etc., for processing by or in connection with the processor 1506. In some implementations, the memory 1510 may include one or more of volatile memory and non-volatile memory (e.g., RAM, ROM, hard disk, optical disk, etc.). It should be understood that the memory 1510 may be a single device or may include multiple types of devices and configurations.

Bus 802 can include a communication bus for transferring data between components of a computing device or between computing devices, a network bus system, or portions thereof, a processor mesh, a combination thereof, etc. In some configurations, the various components of computing device 800 cooperate and communicate via a communication mechanism included in or implemented in association with the bus 802. In some configurations, bus 802 may be a software communication mechanism including and/or facilitating, for example, inter-method communication, local function or procedure calls, remote procedure calls, an object broker (e.g., CORBA), direct socket communication (e.g., TCP/IP sockets) among software modules, UDP broadcasts and receipts, HTTP connections, etc. Further, communication between components of computing device 800 via bus 802 may be secure (e.g., SSH, HTTPS, etc.).

Communication unit 804 may include one or more interface devices (I/F) for wired and/or wireless connectivity among the components of computing device 800 and/or other computing devices, sensors, or other systems configured with similar interface hardware and protocols. For instance, communication unit 804 may include, but is not limited to, various types of known connectivity and interface options, such as Ethernet and WiFi networks, Bluetooth, USB, PCIe, etc. Communication unit 804 may be coupled to the other components of the computing device 800 via bus 802. Communication unit 804 can provide other connections to the network and to other entities of the system in FIG. 4 using various standard communication protocols.

Input device 808 may include any device for inputting information into the computing device 800. In some implementations, input device 808 may include one or more peripheral devices. For example, input device 808 may include a keyboard, a pointing device, microphone, an image/video capture device (e.g., camera), other sensors, a touch-screen display integrated with the output device 814, etc. Output device 814 may be any device capable of outputting information from the computing device 800. Output device 814 may include one or more of a display (LCD, OLED, etc.), a printer, a 3D printer, a haptic device, audio reproduction device, touch-screen display, a remote computing device, indicator lights, etc. In some configurations, output device 814 is a display which may display electronic images and data output by a processor, such as processor 806, of the computing device 800 for presentation to a user, such as through a graphical user interface configured on the display.

While at least one example implementation has been presented in the foregoing detailed description of the technology, it should be appreciated that a vast number of variations may exist. It should also be appreciated that an exemplary implementation or exemplary implementations are examples, and are not intended to limit the scope, applicability, or configuration of the technology in any way. Rather, the foregoing detailed description will provide those skilled in the art with a convenient road map for implementing an example implementation of the technology, it being understood that various modifications may be made in a function and/or arrangement of elements described in an exemplary implementation without departing from the scope of the technology, as set forth in the appended claims and their legal equivalents.

As will be appreciated by one of ordinary skill in the art, various aspects of the present technology may be embodied as a system, method, or computer program product. Accordingly, some aspects of the present technology may take the form of an entirely hardware implementation, an entirely software implementation (including firmware, resident software, micro-code, etc.), or a combination of hardware and software aspects that may all generally be referred to herein as a circuit, module, system, and/or network. Furthermore, various aspects of the present technology may take the form of a computer program product embodied in one or more computer-readable mediums including computer-readable program code embodied thereon.

Any combination of one or more computer-readable mediums may be utilized. A computer-readable medium may be a computer-readable signal medium or a physical computer-readable storage medium. A physical computer readable storage medium may be, for example, but not limited to, an electronic, magnetic, optical, crystal, polymer, electromagnetic, infrared, or semiconductor system, apparatus, or device, etc., or any suitable combination of the foregoing. Non-limiting examples of a physical computer-readable storage medium may include, but are not limited to, an electrical connection including one or more wires, a portable computer diskette, a hard disk, random access memory (RAM), read-only memory (ROM), an erasable programmable read-only memory (EPROM), an electrically erasable programmable read-only memory (EEPROM), a Flash memory, an optical fiber, a compact disk read-only memory (CD-ROM), an optical processor, a magnetic processor, etc., or any suitable combination of the foregoing. In the context of this document, a computer-readable storage medium may be any tangible medium that can contain or store a program or data for use by or in connection with an instruction execution system, apparatus, and/or device.

Computer code embodied on a computer-readable medium may be transmitted using any appropriate medium, including but not limited to, wireless, wired, optical fiber cable, radio frequency (RF), etc., or any suitable combination of the foregoing. Computer code for carrying out operations for aspects of the present technology may be written in any static language, such as the C programming language or other similar programming language. The computer code may execute entirely on a user's computing device, partly on a user's computing device, as a stand-alone software package, partly on a user's computing device and partly on a remote computing device, or entirely on the remote computing device or a server. In the latter scenario, a remote computing device may be connected to a user's computing device through any type of network, or communication system, including, but not limited to, a local area network (LAN) or a wide area network (WAN), Converged Network, or the connection may be made to an external computer (e.g., through the Internet using an Internet Service Provider).

Various aspects of the present technology may be described above with reference to flowchart illustrations and/or block diagrams of methods, apparatus, systems, and computer program products. It will be understood that each block of a flowchart illustration and/or a block diagram, and combinations of blocks in a flowchart illustration and/or block diagram, can be implemented by computer program instructions. These computer program instructions may be provided to a processing device (processor) of a general-purpose computer, special purpose computer, or other programmable data processing apparatus to produce a machine, such that the instructions, which can execute via the processing device or other programmable data processing apparatus, create means for implementing the operations/acts specified in a flowchart and/or block(s) of a block diagram.

Some computer program instructions may also be stored in a computer-readable medium that can direct a computer, other programmable data processing apparatus, or other device(s) to operate in a particular manner, such that the instructions stored in a computer-readable medium to produce an article of manufacture including instructions that implement the operation/act specified in a flowchart and/or block(s) of a block diagram. Some computer program instructions may also be loaded onto a computing device, other programmable data processing apparatus, or other device(s) to cause a series of operational steps to be performed on the computing device, other programmable apparatus or other device(s) to produce a computer-implemented process such that the instructions executed by the computer or other programmable apparatus provide one or more processes for implementing the operation(s)/act(s) specified in a flowchart and/or block(s) of a block diagram.

A flowchart and/or block diagram in the above figures may illustrate an architecture, functionality, and/or operation of possible implementations of apparatus, systems, methods, and/or computer program products according to various aspects of the present technology. In this regard, a block in a flowchart or block diagram may represent a module, segment, or portion of code, which may comprise one or more executable instructions for implementing one or more specified logical functions. It should also be noted that, in some alternative aspects, some functions noted in a block may occur out of an order noted in the figures. For example, two blocks shown in succession may, in fact, be executed substantially concurrently, or blocks may at times be executed in a reverse order, depending upon the operations involved. It will also be noted that a block of a block diagram and/or flowchart illustration or a combination of blocks in a block diagram and/or flowchart illustration, can be implemented by special purpose hardware-based systems that may perform one or more specified operations or acts, or combinations of special purpose hardware and computer instructions.

While one or more aspects of the present technology have been illustrated and discussed in detail, one of ordinary skill in the art will appreciate that modifications and/or adaptations to the various aspects may be made without departing from the scope of the present technology, as set forth in the following claims.

What is claimed is:

1. A system, comprising:
a receptacle configured to receive a pharmaceutical product;
a camera configured to capture image data for at least one image of the pharmaceutical product in the receptacle;
at least one sensor configured to capture sensor data for at least one sensor signal based on the pharmaceutical product in the receptacle;
at least one processor configured to:
receive the image data and the sensor data;
determine at least one visual feature from the image data;
determine at least one sensor feature from the sensor data;
apply at least one machine learning model to the at least one visual feature and the at least one sensor feature to determine a recognition confidence value for a pharmaceutical type; and
return a recognition indicator based on the recognition confidence value for the pharmaceutical type.

2. The system of claim 1, wherein:
the at least one visual feature comprises a plurality of visual features selected from:
a size feature;
a color feature;
a shape feature; and
a marking feature;
the at least one machine learning model comprises a visual classifier model trained to recognize the plurality of visual features corresponding to the pharmaceutical type to generate a visual identifier confidence value; and
the at least one processor is further configured to use the visual identifier confidence value to determine the recognition confidence value.

3. The system of claim 1, wherein:
the at least one sensor comprises a spectrometer configured to capture spectral data based on reflected light from a laser directed at the pharmaceutical product in the receptacle;
the at least one sensor feature comprises at least one spectral feature selected from:
a spectrometer graph; and
a signal-to-noise graph;
the at least one machine learning model comprises at least one classifier model selected from:
a spectral classifier model trained to recognize the spectrometer graph corresponding to the pharmaceutical type to generate a spectral identifier confidence value; and
a signal-to-noise classifier model trained to recognize the signal-to-noise graph corresponding to the pharmaceutical type to generate a signal-to-noise identifier confidence value; and
the at least one processor is further configured to use the spectral identifier confidence value or the signal-to-noise identifier confidence value to determine the recognition confidence value.

4. The system of claim 1, wherein:
the at least one sensor comprises an olfactory sensor configured to capture olfactory data based on response to low-concentration chemicals suspended in air in the receptacle;
the at least one sensor feature comprises at least one olfactory feature corresponding to a presence of at least one volatile chemical present in the pharmaceutical type;
the at least one machine learning model comprises an olfactory classifier model trained to recognize the at least one olfactory feature corresponding to the pharmaceutical type to generate an olfactory identifier confidence value; and
the at least one processor is further configured to use the olfactory identifier confidence value to determine the recognition confidence value.

5. The system of claim 1, wherein:
the at least one sensor comprises an audio sensor configured to capture audio data based on response to vibration of the pharmaceutical product in the receptacle;
the at least one sensor feature comprises at least one audio feature selected from:
a vibration audio signal feature from controlled vibration of the pharmaceutical product; and
a resonance frequency from a vibration frequency sweep of the pharmaceutical product;
the at least one machine learning model comprises an audio classifier model trained to recognize the at least one audio feature corresponding to the pharmaceutical type to generate an audio identifier confidence value; and
the at least one processor is further configured to use the audio identifier confidence value to determine the recognition confidence value.

6. The system of claim 5, further comprising:
a vibration motor configured to controllably vibrate the receptacle using at least one selected frequency, wherein the at least one processor is further configured to selectively initiate the vibration motor to capture the audio data.

7. The system of claim 1, further comprising:
a display in communication with the at least one processor and configured to display, responsive to the return of the recognition indicator by the at least one processor, a graphical user interface comprising the recognition indicator for the pharmaceutical product.

8. The system of claim 1, wherein:
the at least one sensor comprises a plurality of sensors having different sensor types selected from:
an image sensor;
a spectrometer;
an olfactory sensor; and
an audio sensor;
the at least one machine learning model comprises a plurality of classifier models corresponding to the different sensor types;
each classifier model of the plurality of classifier models:
corresponds to a sensor type for that sensor of the plurality of sensors: and
is trained to return an identifier confidence value for the pharmaceutical type and that sensor type; and
the at least one processor is further configured to determine the recognition confidence value based on combining a plurality of identifier confidence values from the plurality of classifier models.

9. The system of claim 1, further comprising:
a multimodal device comprising:
the receptacle;
the camera;
the at least one sensor; and
a control interface for a computing device;
a first computing system at a first location and comprising:
at least one processor configured to execute a pharmaceutical dispensing application for dispensing the pharmaceutical product based on a prescription;
a peripheral interface configured for communication with the control interface of the multimodal device to control capture of the image data and the sensor data; and
a first network interface configured for communication over a network; and
a second computing system at a second location and comprising:
at least one processor configured to execute a verification application for verifying dispensed pharmaceutical product against the prescription;
a second network interface configured for communication over the network; and
a display configured to display, responsive to the return of the recognition indicator by the at least one processor, a graphical user interface comprising the recognition indicator for the pharmaceutical product, wherein the verification application is configured to receive a verification input from a user in response to the display of the recognition indicator.

10. A method, comprising:
receiving, in a receptacle, a pharmaceutical product;
capturing, using a camera, image data for at least one image of the pharmaceutical product in the receptacle;
capturing, using at least one sensor, sensor data for at least one sensor signal based on the pharmaceutical product in the receptacle;
determining, by at least one processor, at least one visual feature from the image data;
determining, by the at least one processor, at least one sensor feature from the sensor data;
determining, by the at least one processor applying at least one machine learning model to the at least one visual feature and the at least one sensor feature, a recognition confidence value for a pharmaceutical type; and
returning, by the at least one processor, a recognition indicator based on the recognition confidence value for the pharmaceutical type.

11. The method of claim 10, further comprising:
determining, by the at least one processor, a visual identifier confidence value, wherein:
the at least one visual feature comprises a plurality of visual features selected from:
a size feature;
a color feature;
a shape feature; and
a marking feature;
the at least one machine learning model comprises a visual classifier model trained to recognize the plurality of visual features corresponding to the pharmaceutical type to generate the visual identifier confidence value; and
determining the recognition confidence value is based on the visual identifier confidence value.

12. The method of claim 10, further comprising:
determining, by the at least one processor, a spectral identifier confidence value or a signal-to-noise identifier confidence value, wherein:
at least one sensor comprises a spectrometer configured to capture spectral data based on reflected light from a laser directed at the pharmaceutical product in the receptacle;
the at least one sensor feature comprises at least one spectral feature selected from:
a spectrometer graph; and
a signal-to-noise graph;
the at least one machine learning model comprises at least one classifier model selected from:
a spectral classifier model trained to recognize the spectrometer graph corresponding to the pharmaceutical type to generate the spectral identifier confidence value; and
a signal-to-noise classifier model trained to recognize the signal-to-noise graph corresponding to the pharmaceutical type to generate the signal-to-noise identifier confidence value; and
determining the recognition confidence value is based on the spectral identifier confidence value or the signal-to-noise identifier confidence value.

13. The method of claim 10, further comprising:
determining, by the at least one processor, an olfactory identifier confidence value, wherein:
the at least one sensor comprises an olfactory sensor configured to capture olfactory data based on response to low-concentration chemicals suspended in air in the receptacle;
the at least one sensor feature comprises at least one olfactory feature corresponding to a presence of at least one volatile chemical present in the pharmaceutical type;
the at least one machine learning model comprises an olfactory classifier model trained to recognize the at least one olfactory feature corresponding to the pharmaceutical type to generate the olfactory identifier confidence value; and
determining the recognition confidence value is based on the olfactory identifier confidence value.

14. The method of claim 10, further comprising:
determining, by the at least one processor, an audio identifier confidence value, wherein:
the at least one sensor comprises an audio sensor configured to capture audio data based on response to vibration of the pharmaceutical product in the receptacle;
the at least one sensor feature comprises at least one audio feature selected from:
a vibration audio signal feature from controlled vibration of the pharmaceutical product; and
a resonance frequency from a vibration frequency sweep of the pharmaceutical product;
the at least one machine learning model comprises an audio classifier model trained to recognize the at least one audio feature corresponding to the pharmaceutical type to generate the audio identifier confidence value; and
determining the recognition confidence value is based on the audio identifier confidence value.

15. The method of claim 14, further comprising:
controllably vibrating, using a vibration motor, the receptacle using at least one selected frequency to initiate capture of the audio data.

16. The method of claim 10, further comprising:
displaying, on a display in communication with the at least on processor and responsive to the return of the recognition indicator by the at least one processor, a graphical user interface comprising the recognition indicator for the pharmaceutical product.

17. The method of claim 10, further comprising:
determining a plurality of identifier confidence values using a plurality of classifier models, wherein:
the at least one sensor comprises a plurality of sensors having different sensor types selected from:
an image sensor;
a spectrometer;
an olfactory sensor; and
an audio sensor;
the at least one machine learning model comprises a plurality of classifier models corresponding to the different sensor types;
each classifier model of the plurality of classifier models:
corresponds to a sensor type for that sensor of the plurality of sensors: and
is trained to return an identifier confidence value for the pharmaceutical type and that sensor type; and
combining the plurality of identifier confidence values from the plurality of classifier models to determine the recognition confidence value.

18. The method of claim 10, further comprising:
receiving, by a first computing system at a first location, a prescription;
dispensing, prior to receiving the pharmaceutical product in the receptacle, the pharmaceutical product based on the prescription;
receiving, by the first computer system and from a multimodal device, the image data and the sensor data, wherein the multimodal device comprises:
the receptacle;
the camera;
the at least one sensor;
receiving, over a network and by a second computing system at a second location, the recognition indicator;
displaying, on a display of the second computing system, a graphical user interface comprising the recognition indicator for the pharmaceutical product; and
receiving, by the second computing system and from a user, a verification input for the pharmaceutical product corresponding to the prescription in response to the display of the recognition indicator.

19. A device, comprising:
a receptacle configured to receive a pharmaceutical product;
a camera configured to capture image data for at least one image of the pharmaceutical product in the receptacle;
at least one sensor configured to capture sensor data for at least one sensor signal based on the pharmaceutical product in the receptacle; and
a control interface to at least one processor configured to:
receive the image data and the sensor data;
determine at least one visual feature from the image data;
determine at least one sensor feature from the sensor data;
apply at least one machine learning model to the at least one visual feature and the at least one sensor feature to determine a recognition confidence value for a pharmaceutical type; and
return a recognition indicator based on the recognition confidence value for the pharmaceutical type.

20. The device of claim 19, wherein:
the at least one sensor comprises a plurality of sensors having different sensor types selected from:
an image sensor;
a spectrometer;
an olfactory sensor; and
an audio sensor;
the at least one machine learning model comprises a plurality of classifier models corresponding to the different sensor types;
each classifier model of the plurality of classifier models:
corresponds to a sensor type for that sensor of the plurality of sensors: and
is trained to return an identifier confidence value for the pharmaceutical type and that sensor type; and
the at least one processor is further configured to determine the recognition confidence value based on combining a plurality of identifier confidence values from the plurality of classifier models.
